(19) United States
(12) Patent Application Publication
(10) Pub. No.: US 2022/0323378 A1
(43) Pub. Date: Oct. 13, 2022

(54) Pharmaceutical Compositions and Methods for Treating Mental Health Disorders and Promoting Neural Plasticity

(71) Applicant: Shawn Joseph, Austin, TX (US)

(72) Inventor: Shawn Joseph, Austin, TX (US)

(21) Appl. No.: 17/713,155

(22) Filed: Apr. 4, 2022

Related U.S. Application Data
(60) Provisional application No. 63/170,486, filed on Apr. 3, 2021.
(60) Provisional application No. 63/173,795, filed on Apr. 12, 2021.
(60) Provisional application No. 63/177,601, filed on Apr. 21, 2021.
(60) Provisional application No. 63/245,592, filed on Sep. 17, 2021.
(60) Provisional application No. 63/247,773, filed on Sep. 23, 2021.
(60) Provisional application No. 63/277,998, filed on Nov. 10, 2021.

(51) Int. Cl. A61K 31/135; A61K 31/485; A61K 31/675; A61K 31/4045

(52) U.S. Cl. CPC A61K 31/135; A61K 31/485; A61K 31/675; A61K 31/4045

(57) ABSTRACT

The present invention relates to methods and compositions useful for treating one or more health conditions/disorders such as brain conditions or disorders. The inventive compositions and methods also promote brain plasticity in patients in need thereof. Health conditions include mental health, pain, aging conditions, and/or disorders such as depression, anxiety, and/or post-traumatic stress disorder (PTSD). The inventive composition contains a serotonergic psychedelic and ketamine that act synergistically, resulting in greater than expected efficacy.

### CROSS-REFERENCE TO RELATED APPLICATIONS

The instant application claims the benefit of U.S. Provisional Patent Application Ser. No. 63/170,486 filed on Apr. 3, 2021, U.S. Provisional Patent Application Ser. No. 63/173,795 filed on Apr. 12, 2021, U.S. Provisional Patent Application Ser. No. 63/177,601 filed on Apr. 21, 2021, U.S. Provisional Patent Application Ser. No. 63/245,592 filed on Sep. 17, 2021, U.S. Provisional Patent Application Ser. No. 63/247,773 filed on Sep. 23, 2021, U.S. Provisional Patent Application Ser. No. 63/277,998 filed on Nov. 10, 2021, each of which is incorporated herein by reference in its entirety.

### BACKGROUND

Mental health conditions and psychiatric disorders such as post-traumatic stress disorder (PTSD), anxiety, and depression are prevalent problems. PTSD, in particular, is highly debilitating and notoriously difficult to treat. PTSD characterizations can include flashbacks, emotional numbness, and insomnia, and may be associated with functional impairments, physical health concerns, and mental health comorbidities, such as depression, with six fold higher risk of suicide. PTSD can result from a catastrophic and threatening event, e.g., a natural disaster, wartime situation, accident, domestic abuse, or violent crime. Symptoms typically develop within three months, but can emerge years after the initial trauma. At some point in their lifetimes, 5-8% of men and 10-14% of women, generally [1].

The treatment of PTSD is extremely challenging, and may include many years of individual and group therapy and medications such as antidepressants, anxiolytic drugs, β-adrenergic antagonists, opiates, or cortisol with variable results. Selective serotonin reuptake inhibitors (SSRIs) are currently recommended as the first-line pharmacotherapy. However, up to 40% of SSRI-treated PTSD patients do not respond and >70% never achieve full remission. The two SSRIs that are approved for PTSD by the United States Food and Drug Administration (FDA), paroxetine and sertraline, have modest effect sizes and limited efficacy in all three clusters of illness: re-experiencing, avoidance and numbing, and hyperarousal [1].

PTSD is particularly prevalent among combat veterans. An estimated 17% of Operation Iraqi Freedom/Operation Enduring Freedom veterans will develop PTSD. A recent Veterans Affairs (VA) clinical trial of the FDA-approved drug, sertraline, failed to show efficacy in a group of patients with predominantly combat-related PTSD. The severity and significance of lack of SSRI efficacy, especially in light of the observed relationship between trauma exposure and increased rates of disability, unemployment, and social assistance highlights the urgent need for novel pharmacological interventions targeting the core pathophysiology of PTSD [1].

Ketamine is an antagonist of NMDA-type glutamate receptors. Ketamine exhibits anesthetic properties at high doses, e.g., doses of ^{˜}2 mg/kg, and analgesic properties at subanesthetic doses. Ketamine is considered safe with minimal to moderate side effects [1].

Ketamine can serve as a fast acting antidepressant [2]. However, the effects may be short-lived [3-5].

This creates disadvantages when ketamine is used. In one example, because ketamine can lose efficacy over time, it can cause patients to return for additional treatment [6-7]. Repeat visits for repeat treatment thereby increases the chance or incidence of side effects associated with treatment [9].

Thus, ketamine causes a challenge in that it is insufficient as a solution on its own [6-7] [10-11].

There is a need in the art for improved methods for the treatment of PTSD and other brain disorders or conditions. The present disclosure describes compositions, and methods of making and/or using thereof, to treat PTSD and other brain disorders or conditions.

### BRIEF SUMMARY OF THE INVENTION

The present invention relates to methods and compositions for the treatment of one or more health conditions including, mental health, pain, aging conditions, and/or disorders such as depression, anxiety, and/or post-traumatic stress disorder (PTSD).

In an aspect, the present invention is a composition, method of making the same, and/or method of using the same, wherein the composition comprises 1) a serotonergic psychedelic and/or a serotonergic psychedelic derivative and 2) ketamine and/or a ketamine derivative. In some embodiments, the present invention is for increasing neural plasticity of neuronal cells, and/or for treating a brain condition or disorder.

In an aspect, provided herein is a method of increasing neural plasticity. The method includes administering 1) a serotonergic psychedelic, an analogue or derivative thereof, and 2) ketamine, an analogue or derivative thereof, or any combination of (1) and (2), in an amount sufficient to increase neural plasticity of the neuronal cell.

In an aspect, provided herein is a method of increasing neural plasticity. The method includes contacting a neuronal cell with a composition comprising compounds including a serotonergic psychedelic, or a derivative thereof, and ketamine, or a derivative thereof, in an amount sufficient to increase neural plasticity of the neuronal cell, wherein the composition produces a maximum number of dendritic crossings with an increase of greater than 1.0 fold by a Sholl Analysis.

In aspect, provided herein is a method of treating a brain condition or disorder. The method includes administering to a subject in need thereof a therapeutically effective amount of a composition comprising a serotonergic psychedelic, or a derivative thereof, and ketamine, or a derivative thereof, thereby treating the brain condition or disorder, wherein the composition comprising a serotonergic psychedelic, or a derivative thereof, and ketamine, or a derivative thereof, increases neural plasticity of the neuronal cell.

In some embodiments, a method of treatment comprises the administration of a therapeutically effective amount of a composition comprising psilocybin, a prodrug of psilocybin, an active metabolite of psilocybin, or a prodrug of an active metabolite of psilocybin, and ketamine, to a subject in need thereof as described herein.

In some embodiments, a method of treatment comprises the administration of a therapeutically effective amount of a composition comprising psilocybin and ketamine as described herein. In some embodiments, a method of treatment comprises the administration of a therapeutically effective amount of a composition comprising psilocin and ketamine as described herein.

Some embodiments comprise a composition comprising psilocybin, a prodrug of psilocybin, an active metabolite of psilocybin, or a prodrug of an active metabolite of psilocybin, and ketamine for use in the treatment of an indication as described herein. Some embodiments comprise a composition comprising psilocybin and ketamine for use in the treatment of an indication as described herein. Some embodiments comprise a composition comprising psilocin and ketamine for use in the treatment of an indication as described herein.

Some embodiments comprise the use of a composition comprising psilocybin, a prodrug of psilocybin, an active metabolite of psilocybin, or a prodrug of an active metabolite of psilocybin, and ketamine in the manufacture of a medicament for the treatment of an indication as described herein. Novel polymorphs and hydrates of psilocybin, along with the preparation and formulations thereof are disclosed in U.S Application No. US2019/0119310 A1, which is incorporated by reference herein in its entirety. US2019/0119310 discloses a number of formulations and the challenges of formulating psilocybin due to e.g. its hygroscopicity and poor flow characteristics. US2019/01 19310 also discloses the importance of a controlled aqueous crystallisation process. WO2020212952A1, which is incorporated by reference herein in its entirety, discloses various aspects relating to preparations and formulations relating to psilocybin. U.S. Pat. No. 8,785,500B2, which is incorporated by reference herein in its entirety, discloses various aspects relating to preparations and formulations relating to ketamine. US2013/0236573A1, which is incorporated by reference herein in its entirety, discloses various aspects relating to preparations and formulations relating to ketamine. All other papers cited herein are incorporated by reference herein in their entirety.

### BRIEF DESCRIPTION OF DRAWINGS

FIG. 1 is a diagram showing the structures of ketamine and psilocin side-by-side.

FIG. 2 is a diagram showing the structures of some classical hallucinogens side-by-side.

FIG. 3A is a diagram of an hSERT structure isolated using x-ray crystallography.

FIG. 3B is a diagram of an hSERT structure predicted by Google DeepMind AlphaFold 2 artificial intelligence software.

FIG. 3C is a diagram showing the predicted aligned error of the structure shown in FIG. 3B.

FIG. 4 is a schematic view of an example test object in two different poses relative to a target object, according to an embodiment.

FIG. 5 is a diagram of a serotonin hydrophilic pocket around Ala173 and Ser438 in hSERT.

FIG. 6 is a diagram showing an example docking pose of psilocin in hSERT.

FIG. 7 is a diagram showing a two-layer neural network model.

FIG. 8 is a diagram showing a two-layer neural network model that associates an input pattern with an output pattern.

### DETAILED DESCRIPTION

The present invention relates to methods and compositions for the treatment of one or more health conditions including, mental health, pain, aging conditions, and/or disorders such as depression, anxiety, and/or post-traumatic stress disorder (PTSD) that may be associated with neuron atrophy. More particularly, some embodiments of the invention relate to intranasal, intravenous, or transdermal administration of composition comprising a serotonergic psychedelic such as psilocybin or a psilocybin derivative and ketamine or a ketamine derivative to treat one or more health or mental health conditions.

In some embodiments, the present invention is a composition, method of making the same, and/or method of using the same, wherein the composition comprises 1) a serotonergic psychedelic and/or a serotonergic psychedelic derivative and 2) ketamine and/or a ketamine derivative. In some embodiments, the present invention is for increasing neural plasticity of neuronal cells, and/or for treating a brain condition or disorder.

Depression is one of the most prevalent and disabling diseases in the world [2], and one that is often left untreated.

Ketamine can serve as a fast acting antidepressant [2]. However, the effects may be short-lived [3-5].

This creates disadvantages when ketamine is used. In one example, because ketamine can lose efficacy over time, it can cause patients to return for additional treatment [6-7]. Repeat visits for repeat treatment thereby increases the chance or incidence of side effects associated with treatment [9].

Thus, ketamine causes a challenge in that it is insufficient as a solution on its own [6-7] [10-11].

In some cases, a patient may already be receiving, or planning to receive, a treatment via nasal or intravenous administration of ketamine, for example. In such cases, it would be trivial to provide, instead, or within, a composition wherein ketamine is included, without requiring additional inconvenience or pain for the patient, providing practical advantages.

Cooperative and/or synergistic effects are gained by the use of compositions described herein.

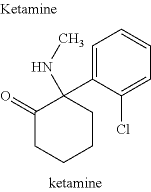 [SMILES: CNC1(c2ccccc2Cl)CCCCC1=O]

Ketamine shows rapid antidepressant activity in depression patients that lasts up to weeks via mechanisms including alterations in AMPA (α-amino-3-hydroxy-5-methyl-4-isoxazolepropionic acid) receptors, Brain-derived neurotrophic factor (BDNF), mammalian target of rapamycin (mTOR), and glycogen synthase kinase-3 (GSK3), the formation of new dendritic spines and synapses in the prefrontal cortex, and/or acceleration of hippocampal granule cell maturation/integration into hippocampal networks [12]. Further, ketamine blockades the N-methyl-D-aspartate (NMDA) receptor/channel [13]. The given systems are highly complex with interactions occurring at all levels [13].

Especially relevant, ketamine enhances BDNF/synaptic protein levels and mTOR stimulation, rapidly increasing synaptogenesis, including increasing density and function of spine synapses, in the prefrontal cortex (PFC) [13-14]. Ketamine rapidly thereby causes a functional connection, reconnection, and/or interaction of neurons [14].

Serotonergic Psychedelics

Serotonergic psychedelics such as psilocybin, psilocybin derivatives, LSD, DMT, and DOI increase neuritogenesis and/or spinogenesis by stimulation and/or interaction with TrkB (tropomyosin receptor kinase B), mTOR, and 5-HT2A pathways [15-16]. However, while downstream effects of ketamine and serotonergic psychedelics, such as mTOR stimulation, may have features in common and/or similarities, the mechanisms differ. For example, many serotonergic psychedelics are not NMDA inhibiting. These distinctions create a therapeutic opportunity.

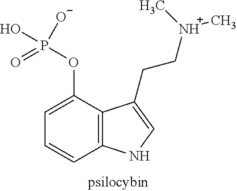 [SMILES: C[NH+](C)CCc1c[nH]c2cccc(OP(=O)([O-])O)c12]

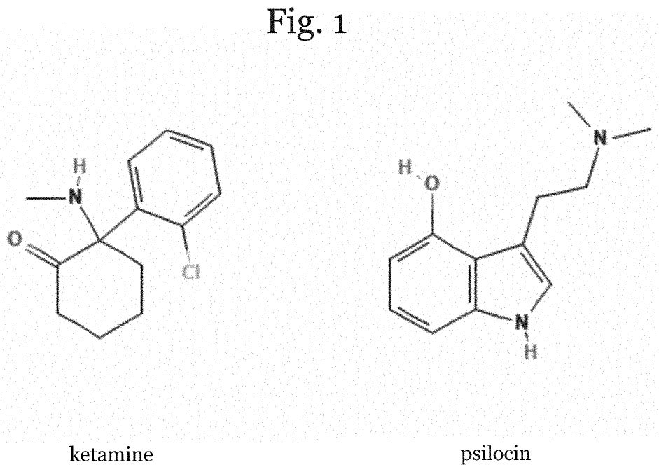

FIG. 1 is a diagram showing the structures of ketamine and psilocybin side-by-side.

Cooperativity of Ketamine and Serotonergic Psychedelics

First, in one example, since ketamine and serotonergic psychedelics influence mTOR via different avenues, the former via NMDA receptor interactions and the latter via 5-HT2A and/or TrkB receptor interactions, a cooperative, concerted, and/or amplified effect on mTOR can be gained without, for example in some cases, increasing a ketamine dose beyond a preferable low level or limit. This can prevent or stem approaching a need for coming nearer to addictive levels of ketamine. That is, ketamine may be considered limited in its therapeutic use or scope because increasing dosage increases addiction probability.

This effectively limits ketamine's power as a therapeutic to promote neurogenesis and/or synaptogenesis. But in combination with a non-addictive serotonergic psychedelic exerting an alternate force on the mTOR pathway, a concerted and amplified action can be achieved to remove the ceiling that ketamine therapy suffers.

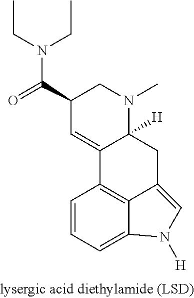 [SMILES: CCN(CC)C(=O)[C@@H]1C=C2c3cccc4[nH]cc(c34)C[C@@H]2N(C)C1]

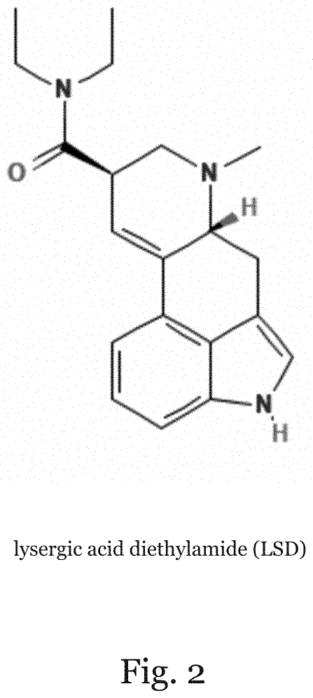

FIG. 2 is a diagram showing the structures of lysergic acid diethylamide (LSD).

Human Serotonin Transporter (hSERT)

Serotonin (5-HT) plays an important regulatory role in mood and other calibrating physiological functions such as appetite and sleep modulation. The 5-HT presence is carefully controlled by the human serotonin transporter (hSERT) which acts by removing/re-uptaking free 5-HT. The hSERT is a member of the neurotransmitter-sodium symporter (NSS) family that is present in the presynaptic membrane. This key role then renders hSERT a potential target for both natural and/or synthetic drugs as therapies for mood-related disorders, for example [17].

Computational/Molecular Docking Studies Using Google DeepMind AlphaFold 2 and Protein Data Bank Structures

Studies of hallucinogenic compounds can be challenging because of the controlled nature of the substances. For example, Drug Enforcement Agency (DEA) clearance, which can be tedious, is needed to carry out lab studies.

Computational studies involving controlled substances therefore currently possess a great advantage over lab studies. Computational studies such as those described in U.S. Pat. No. 11,080,570 B, which is incorporated by reference in its entirety, are applied herein.

In some embodiments, x-ray crystallographic structures for target objects are obtained from the Protein Data Bank.

Protein Data Bank Structures

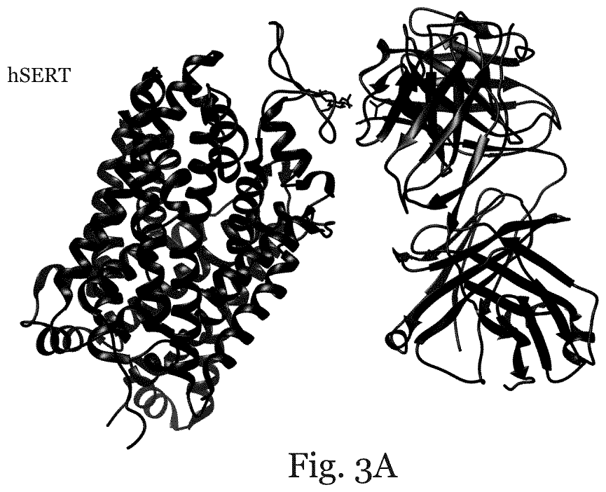

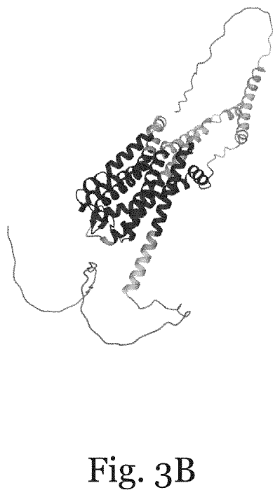

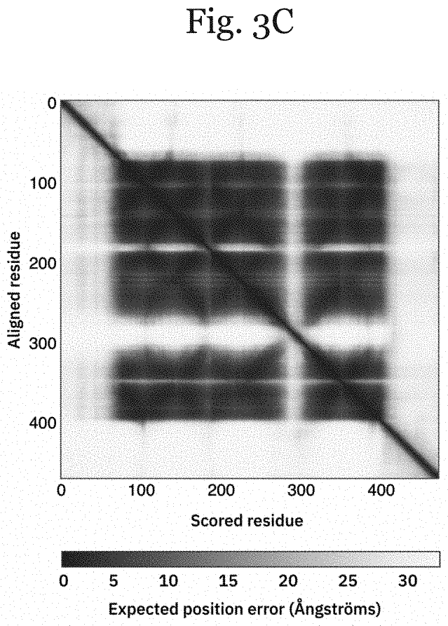

FIG. 3A is a diagram of an hSERT structure isolated using x-ray crystallography [18].

Google DeepMind AlphaFold 2 Structures

In some embodiments, AlphaFold 2 is used to predict target object structures. AlphaFold 2 is an artificial intelligence (AI) program developed by Alphabets's/Google's DeepMind which performs predictions of protein structure.

FIG. 3B is a diagram of an hSERT structure predicted by Google DeepMind AlphaFold 2 artificial intelligence software [19].

The structure of hSERT was obtained from both AlphaFold v 2.0.

Protein 5-hydroxytryptamine receptor 2A

Gene HTR2A

Source organism Homo sapiens

Predicted aligned error

FIG. 3C is a diagram showing the predicted aligned error of the structure shown in FIG. 3B.

The color and/or shade at position (x, y) indicates AlphaFold's expected position error at residue x, when the predicted and true structures are aligned on residue y.

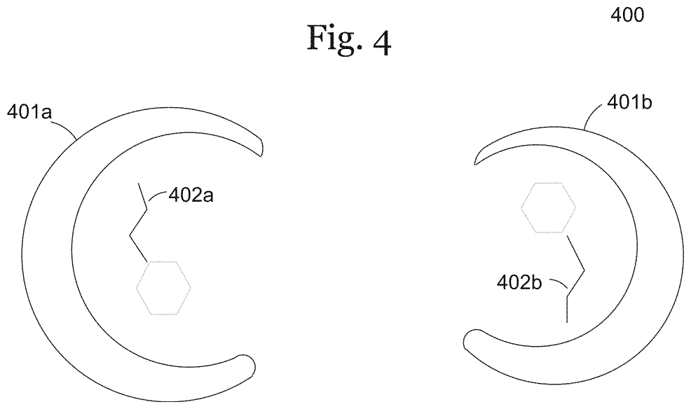

FIG. 4 is a schematic view of an example test object (402a and 402b) in two different poses relative to a target object (401a and 401b), according to an embodiment.

Test objects 402a-402b are modeled with the target objects 401a-401b in each pose of a plurality of different poses. In some embodiments, the target object is a polymer with an active site such as hSERT, the test object is a chemical compound such as ketamine, a serotonergic psychedelic, a serotonergic psychedelic derivative, hallucinogen, and/or hallucinogen derivative, and the modeling comprises docking the test object into the active site of the polymer. In some embodiments, the test object 402a-402b is docked onto the target object 401a-401b a plurality of times to form a plurality of poses. In some embodiments, the test object 402a-402b is docked onto the target object twice, three times, four times, five or more times, ten or more times, fifty or more times, 100 or more times, or a 1000 or more times. Each such docking represents a different pose of the test object 402a-402b docked onto the target object. In some embodiments, the target object is a polymer with an active site and the test object 402a-402b is docked into the active site in each of plurality of different ways, each such way representing a different pose.

Computational/Molecular Docking Studies

In some embodiments, test objects 402a-402b are docked by either random pose generation techniques, or by biased pose generation. In some embodiments, test objects 402a-402b are docked by Markov chain Monte Carlo sampling. In some embodiments, such sampling allows the full flexibility of test objects in the docking calculations and a scoring function that is the sum of the interaction energy between the training (or test) object and the target object as well as the conformational energy of the training (or test) object. See, for example, Liu and Wang, 1999, “MCDOCK: A Monte Carlo simulation approach to the molecular docking problem,” Journal of Computer-Aided Molecular Design 13, 435-451, which is hereby incorporated by reference.

In some embodiments, algorithms such as DOCK (Shoichet, Bodian, and Kuntz, 1992, “Molecular docking using shape descriptors,” Journal of Computational Chemistry 13(3), pp. 380-397; and Knegtel, Kuntz, and Oshiro, 1997 “Molecular docking to ensembles of protein structures,” Journal of Molecular Biology 266, pp. 424-440, each of which is hereby incorporated by reference) are used to find a plurality of poses for each of the test objects 402a-402b and/or training objects against each of the target objects 401a-401b. Such algorithms model the target object and the test (or training) object as rigid bodies. The docked conformation is searched using surface complementary to find poses.

In some embodiments, algorithms such as AutoDOCK (Morris et al., 2009, “AutoDock4 and AutoDockTools4: Automated Docking with Selective Receptor Flexibility,” J. Comput. Chem. 30(16), pp. 2785-2791; Sotriffer et al., 2000, “Automated docking of ligands to antibodies: methods and applications,” Methods: A Companion to Methods in Enzymology 20, pp. 280-291; and “Morris et al., 1998, “Automated Docking Using a Lamarckian Genetic Algorithm and Empirical Binding Free Energy Function,” Journal of Computational Chemistry 19: pp. 1639-1662, each of which is hereby incorporated by reference) are used to find a plurality of poses for each of the test objects 402a-402b and/or training objects against each of the target objects 401a-401b. AutoDOCK uses a kinematic model of the ligand and supports Monte Carlo, simulated annealing, the Lamarckian Genetic Algorithm, and Genetic algorithms. Accordingly, in some embodiments the plurality of different poses (for a given test object-target object pair or a given training object-test object pair) are obtained by Markov chain Monte Carlo sampling, simulated annealing, Lamarckian Genetic Algorithms, or genetic algorithms, using a docking scoring function.

In some embodiments, algorithms such as FlexX (Rarey et al., 1996, “A Fast Flexible Docking Method Using an Incremental Construction Algorithm,” Journal of Molecular Biology 261, pp. 470-489, which is hereby incorporated by reference) are used to find a plurality of poses for each of the test objects 402a-402b and/or training objects against each of the target objects 401a-401b. FlexX does an incremental construction of the test object 402a-402b and/or training object at the active site of a target object 401a-401b using a greedy algorithm. Accordingly, in some embodiments the plurality of different poses (for a given test object-target object pair or a given training object-test object pair) are obtained by a greedy algorithm.

In some embodiments, algorithms such as GOLD (Jones et al., 1997, “Development and Validation of a Genetic Algorithm for flexible Docking,” Journal Molecular Biology 267, pp. 727-748, which is hereby incorporated by reference) are used to find a plurality of poses for each of the test objects 402a-402b and/or training objects against each of the target objects 401a-401b. GOLD stands for Genetic Optimization for Ligand Docking. GOLD builds a genetically optimized hydrogen bonding network between the test object 402a-402b and/or training object and the target object 401a-401b.

In some embodiments, the modeling comprises performing a molecular dynamics run of the target object and the test object. During the molecular dynamics run, the atoms of the target object and the test object are allowed to interact for a fixed period of time, giving a view of the dynamical evolution of the system. The trajectory of atoms in the target object and the test object (or training object) are determined by numerically solving Newton's equations of motion for a system of interacting particles, where forces between the particles and their potential energies are calculated using interatomic potentials or molecular mechanics force fields. See Alder and Wainwright, 1959, “Studies in Molecular Dynamics. I. General Method,”. J. Chem. Phys. 31 (2): 459; and Bibcode, 1959, J. Ch. Ph. 31, 459A, doi:10.1063/1.1730376, each of which is hereby incorporated by reference. Thus, in this way, the molecular dynamics run produces a trajectory of the target object and the test object together overtime. This trajectory comprises the trajectory of the atoms in the target object and the test object. In some embodiments, a subset of the plurality of different poses is obtained by taking snapshots of this trajectory over a period of time. In some embodiments, poses are obtained from snapshots of several different trajectories, where each trajectory comprise a different molecular dynamics run of the target object interacting with the test object. In some embodiments, prior to a molecular dynamics run, a test object (or a training object) is first docketed into an active site of the target object using a docking technique.

Regardless of what modeling method is used, what is achieved for any given test object 402a-402b/training object-target object 401a-401b pair is a diverse set of poses of the test/training object with the target object with the expectation that one or more of the poses is close enough to the naturally occurring pose to demonstrate some of the relevant intermolecular interactions between the given test object 402a-402b/training object-target object 401a-401b pair.

Molecular docking (MD) studies have been performed identifying a serotonin hydrophilic pocket around Ala173 and Ser438 in hSERT [18], hereafter referred to as the “First Binding Site”.

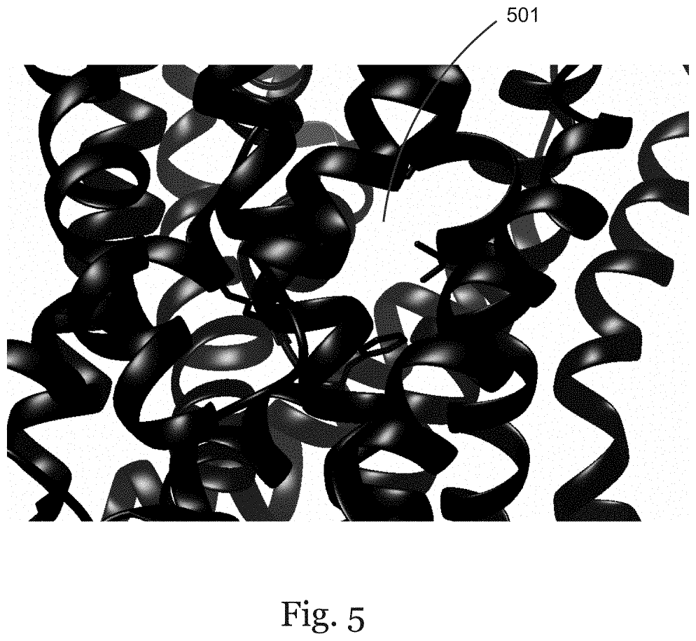

FIG. 5 is a diagram of a serotonin hydrophilic pocket 501 around Ala173 and Ser438 in hSERT.

Molecular docking and binding studies were performed using AutoDock [20], AutoDock Vina [20], and other tools. Chimera and other tools were used to visualize and prepare structures and ligands retrieved from the Protein Data Bank. Both blind docking and bounded docking were performed at the First Binding Site and surrounding areas.

One advantage of the molecular docking studies performed is that various binding sites of the hSERT and binding affinities for various ligands could be studied.

Multiple ligand studies were also performed using a sequential docking procedure. These procedures advantageously allowed study of receptor-compound complexes.

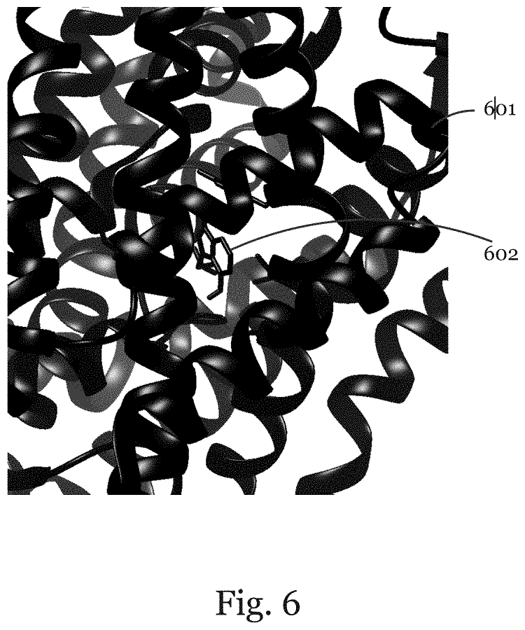

FIG. 6 is a diagram showing an example docking pose of psilocin in hSERT.

### EXAMPLES

The present disclosure is described further below in examples which are intended to further describe the invention without limiting its scope.

Molecular Docking/Binding Affinity Controlled Studies:

These examples establish baseline binding energies/affinities for various constituents including classical hallucinogens, serotonergic psychedelics, and ketamine at the First Binding Site which may act as an orthosteric and/or allosteric site in hSERT and/or at which steric interactions may occur.

Binding energies/affinities for each compound are compared to draw inferences regarding binding competition, behaviors, and/or kinetics of each constituent at the binding site in various compositions and/or solutions of the constituents.

### Example 1/Model 1: Ketamine and Classical Hallucinogens/Serotonergic Psychedelics at First Binding Site in hSERT

In a first experiment, ketamine was docked at the First Binding Site in an hSERT target object obtained and as described herein. An average binding affinity of −6.433 kcal/mol was observed.

TABLE 1

| | Binding | | |
|---|---|---|---|
| Ligand - ketamine | Affinity | | |
| (Receptor = hSERT) | (kcal/mol) | rmsd/ub | rmsd/lb |
| 5i6z_3821_uff_E = | −7.5 | 0 | 0 |
| 1553.83_uff_E = 1551.88 | | | |
| 5i6z_3821_uff_E = | −6.7 | 4.292 | 3.129 |
| 1553.83_uff_E = 1551.88 | | | |
| 5i6z_3821_uff_E = | −6.7 | 4.489 | 2.771 |
| 1553.83_uff_E = 1551.88 | | | |
| 5i6z_3821_uff_E = | −6.6 | 4.494 | 3.194 |
| 1553.83_uff_E = 1551.88 | | | |
| 5i6z_3821_uff_E = | −6.3 | 4.265 | 2.797 |
| 1553.83_uff_E = 1551.88 | | | |
| 5i6z_3821_uff_E = | −6.2 | 4.201 | 3.064 |
| 1553.83_uff_E = 1551.88 | | | |
| 5i6z_3821_uff_E = | −6.2 | 18.054 | 16.081 |
| 1553.83_uff_E = 1551.88 | | | |
| 5i6z_3821_uff_E = | −5.9 | 18.32 | 15.771 |
| 1553.83_uff_E = 1551.88 | | | |
| 5i6z_3821_uff_E = | −5.8 | 7.279 | 5.211 |
| 1553.83_uff_E = 1551.88 | | | |

Average Binding Affinity=−6.433 kcal/mol

### Example 2/Model 2: Ketamine at First Binding Site in hSERT at First Binding Site in First Psilocin-hSERT Complex

In a second experiment, ketamine was docked at the First Binding Site in hSERT after psilocin was docked with hSERT. While one would expect ketamine binding affinity to be disaffected, or worsened, in the presence of psilocin (due to, for example, steric interference and/or competition for the binding site), instead an average binding affinity of −6.811 kcal/mol was observed.

TABLE 2

| | Binding | | |
|---|---|---|---|
| Ligand - ketamine | Affinity | | |
| (Receptor = psilocin + hSERT) | (kcal/mol) | rmsd/ub | rmsd/lb |
| 5i6z-psilo-model2- | −7.5 | 0 | 0 |
| merged_3821_uff_E = | | | |
| 1553.83_uff_E = 1551.88 | | | |
| 5i6z-psilo-model2- | −7.3 | 4.913 | 2.138 |
| merged_3821_uff_E = | | | |
| 1553.83_uff_E = 1551.88 | | | |
| 5i6z-psilo-model2- | −7 | 4.638 | 2.682 |
| merged_3821_uff_E = | | | |
| 1553.83_uff_E = 1551.88 | | | |
| 5i6z-psilo-model2- | −6.7 | 4.575 | 2.871 |
| merged_3821_uff_E = | | | |
| 1553.83_uff_E = 1551.88 | | | |
| 5i6z-psilo-model2- | −6.7 | 4.301 | 3.15 |
| merged_3821_uff_E = | | | |
| 1553.83_uff_E = 1551.88 | | | |
| 5i6z-psilo-model2- | −6.6 | 4.444 | 3.179 |
| merged_3821_uff_E = | | | |
| 1553.83_uff_E = 1551.88 | | | |
| 5i6z-psilo-model2- | −6.5 | 5.478 | 4.246 |
| merged_3821_uff_E = | | | |
| 1553.83_uff_E = 1551.88 | | | |
| 5i6z-psilo-model2- | −6.5 | 5.222 | 2.962 |
| merged_3821_uff_E = | | | |
| 1553.83_uff_E = 1551.88 | | | |
| 5i6z-psilo-model2- | −6.5 | 4.225 | 2.748 |
| merged_3821_uff_E = | | | |
| 1553.83_uff_E = 1551.88 | | | |

Average Binding Affinity=−6.811 kcal/mol

Difference Scores Calculations

Difference score calculations were performed to confirm that the results were statistically significant.

Model 1

N₁: 9

df₁=N−1=9−1=8

M₁: −6.43

SS₁: 2.12

s²₁=SS₁/(N−1)=2.12/(9−1)=0.26

Model 2

N₂: 9

df₂=N−1=9−1=8

M₂: −6.81

SS₂: 1.11

s²₂=SS₂/(N−1)=1.11/(9−1)=0.14

T-Value Calculation

s²ₚ=((df₁/(df₁+df₂))*s²₁)+((df₂/(df₂+df₂))*s²₂)=((8/16)*0.26)+((8/16)*0.14)=0.2

s²_{M1}=s²ₚ/N₁=0.2/9=0.02

s²_{M2}=s²ₚ/N₂=0.2/9=0.02

t=(M₁−M₂)/√(s²_{M1}+s²_{M2})=0.38/√0.04=1.78

The t-value is 1.78392. The p-value is 0.046706. The result is significant at p<0.05.

Concerted Activity and Network Effects

Some estimates provide that neocortical neurons possess an average of 7,000 synaptic connections each [22]. Loss of neurons or connections can have profound effects [22]. Similarly, gain or return of neurons and/or connections can have profound effects. Since each additional neuron can possess so many thousands of synaptic connections, neuron addition to a network or neurons can provide exponential returns.

Ketamine therapy can be a powerful tool as it can modify the number and function of synaptic connections. Since serotonergic psychedelic compounds can also modify the number and function of synaptic connections, a combination or concerted therapy of the two causes a compounding, complex interaction between number and function of synaptic connections from both causes because network interactions in synaptic structures can be exponential. That is, a “network effect” is obtained.

In some embodiments, initially, ketamine in a composition acts as an accelerant of synaptic integration and maturation of neurons already present. Neuron growth may have been stimulated by one or both of ketamine and/or serotonergic psychedelics. A mixture of one or more serotonergic psychedelics in a composition may act to promote longer term neuron growth. Further therapy with the composition may continue a cycle of synaptogenesis promoted by ketamine in the composition. In some embodiments, in each subsequent treatment round with the composition, the composition used may comprise a reduced ketamine to serotonergic psychedelic proportion, for example, to break away from the use of the addictive ketamine.

In some embodiments, ketamine and serotonergic psychedelics may be used separately and/or sequentially. For example, an initial treatment may comprise a serotonergic psychedelic to trigger neurogenesis, while ketamine or psychedelic+ketamine therapy may be used a period of time subsequent to the initial treatment to trigger synaptogenesis of neuron growth. In another embodiment, ketamine therapy may be used initially to trigger synaptogenesis of existing neuron growth and to provide rapid antidepressant activity. A subsequent treatment may comprise a serotonergic psychedelic or a composition of a serotonergic psychedelic and ketamine, as described herein.

Computational/Artificial Neural Network Modeling

Synaptic integration and excitability can be difficult to study. However, simplified models may be used to demonstrate or simulate these activities, for example by applying artificial neural network (ANN) modeling computational techniques [23].

One simple and computationally powerful model has been used that represents a neuron as a binary unit [24]. This simplification of the brain allows neuroscientists to create comparisons between complex neural systems such as the brain and simpler computing systems.

These computational models propose that artificial neurons integrate inputs from presynaptic neurons according to the relative strengths of individual synaptic connections, and fires only if the net input exceeds a threshold value:

nᵢ(t+1)=Θ(Σⱼwᵢⱼnⱼ(t)−μᵢ)

where nᵢ represents the activation (or “firing”) state (0 or 1) of neuron-like unit i at time t. Θ(x) is a thresholding function that equals 1 if x≥μᵢ (the threshold at which unit i fires an impulse), and 0 if x<μᵢ. The weight wᵢⱼ represents the synaptic strength of the synapse from unit j onto unit i [23-24].

Arrays of units described above have been used to develop complex associative memory models that could store multiple patterns, or associations, superimposed across the same synapses:

$${w}_{ij} = \frac{1}{N} {\sum }_{μ = 1}^{p} {ξ}_{i} {ξ}_{j}$$

where N is the number of units in the network, p is the number of associations stored and ζ represents a pattern composed of an array of bits, and wherein the learning in the network is Hebbian [23-25].

“Let us assume then that the persistence or repetition of a reverberatory activity (or “trace”) tends to induce lasting cellular changes that add to its stability. The assumption can be precisely stated as follows: When an axon of cell A is near enough to excite a cell B and repeatedly or persistently takes part in firing it, some growth process or metabolic change takes place in one or both cells such that A's efficiency, as one of the cells firing B, is increased [23-25]”

Correlation matrices have been used to illustrate principles such as distributive associated memory in hippocampal circuitry.

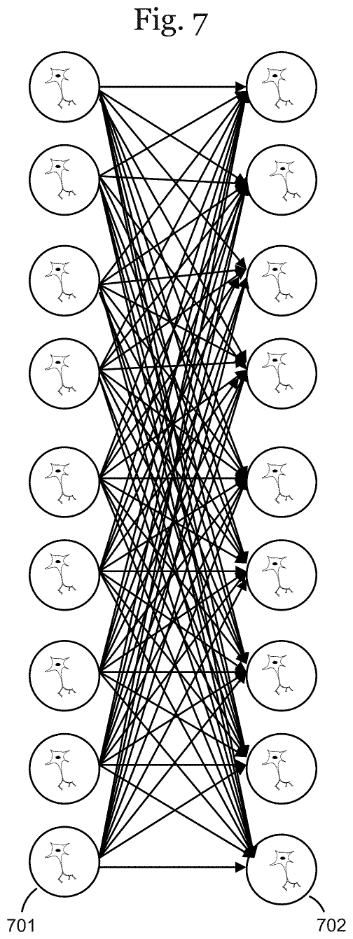

FIG. 7 is a diagram showing a two-layer neural network model.

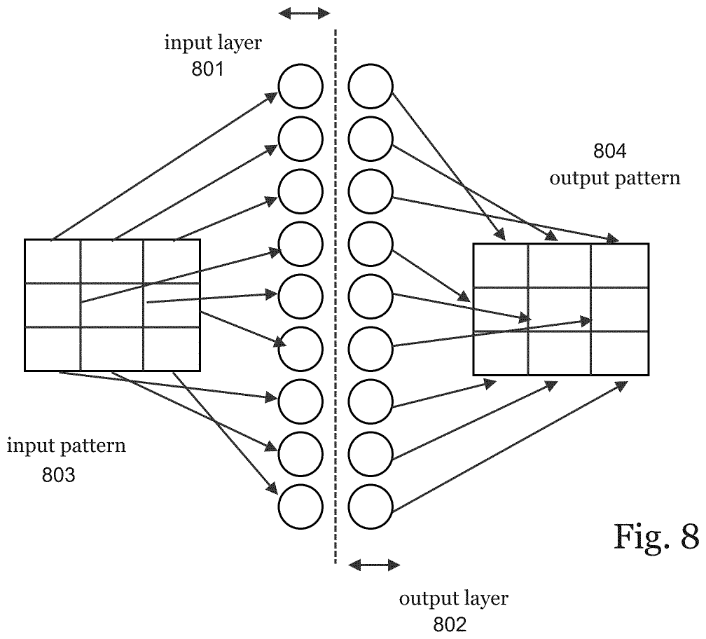

FIG. 8 is a diagram showing a two-layer neural network model that associates an input pattern with an output pattern.

Experiment 1

Correlation matrices were used similarly, and built upon to model complex neural network processes such as to simulate the effects of simple neurogenesis, simple synaptogenesis, and combined neurogenesis/synaptogenesis.

Initial State:

In one example, a 9×9 correlation matrix showing an input layer for an input pattern and an output layer for an output pattern:

| | | | | | | | | |
|---|---|---|---|---|---|---|---|---|
| 1 | 0 | 0 | 0 | 1 | 0 | 0 | 1 | 1 |
| 0 | 0 | 0 | 0 | 0 | 0 | 0 | 0 | 0 |
| 1 | 0 | 0 | 0 | 1 | 0 | 0 | 1 | 1 |
| 1 | 0 | 0 | 0 | 1 | 0 | 0 | 1 | 1 |
| 1 | 0 | 0 | 0 | 1 | 0 | 0 | 1 | 1 |
| 0 | 0 | 0 | 0 | 0 | 0 | 0 | 0 | 0 |
| 0 | 0 | 0 | 0 | 0 | 0 | 0 | 0 | 0 |
| 0 | 0 | 0 | 0 | 0 | 0 | 0 | 0 | 0 |
| 1 | 0 | 0 | 0 | 1 | 0 | 0 | 1 | 1 |

Output of a thresholding function when simulating recall of the correct output pattern when the artificial nerve net is prompted with a trained input pattern provides:

| | | | | | | | | |
|---|---|---|---|---|---|---|---|---|
| 1 | 0 | 0 | 0 | 1 | 0 | 0 | 1 | 1 |

The above state represents 100% recall of the output pattern.

Simulation of Atrophy/Trauma Event:

A trauma or atrophy event can be simulated by, for example, removing a set of nodes or neurons and/or removing synapses:

| | | | | | | | |
|---|---|---|---|---|---|---|---|
| 1 | 0 | 0 | 0 | 1 | 0 | 0 | 1 |
| 0 | 0 | 0 | 0 | 0 | 0 | 0 | 0 |
| 1 | 0 | 0 | 0 | 1 | 0 | 0 | 1 |
| 1 | 0 | 0 | 0 | 1 | 0 | 0 | 1 |
| 1 | 0 | 0 | 0 | | | | |
| 0 | 0 | 0 | 0 | | | | |
| 0 | 0 | 0 | 0 | | | | |
| 0 | 0 | 0 | 0 | | | | |
| 1 | 0 | 0 | 0 | | | | |

Naturally, one can inuit the inability of the given recall simulation to after the trauma event to meet the efficiencies of abilities of the original network. Output of a thresholding function when simulating recall of the correct output pattern when the artificial nerve net is prompted with a trained input pattern provides:

| | | | | | | | | |
|---|---|---|---|---|---|---|---|---|
| 1 | 0 | 0 | 0 | 0 | 0 | 0 | 0 | 0 |

The above state represents 25% recall of the output pattern.

Model 1: Neurogenesis Absent Synaptogenesis after Atrophy or Trauma

Recovery from a trauma event begins when new nodes are created to simulate the growth of new neurons. However, unless a synaptogenesis between these new members is accelerated, recovery to the original network state cannot be achieved.

| | | | | | | | | |
|---|---|---|---|---|---|---|---|---|
| 1 | 0 | 0 | 0 | 1 | 0 | 0 | 1 | 1 |
| 0 | 0 | 0 | 0 | 0 | 0 | 0 | 0 | |
| 1 | 0 | 0 | 0 | 1 | 0 | 0 | 1 | |
| 1 | 0 | 0 | 0 | 1 | 0 | 0 | 1 | |
| 1 | 0 | 0 | 0 | | | | | |
| 0 | 0 | 0 | 0 | | | | | |
| 0 | 0 | 0 | 0 | | | | | |
| 0 | 0 | 0 | 0 | | | | | |
| 1 | 0 | 0 | 0 | | | | | |

Output of a thresholding function when simulating recall of the correct output pattern when the artificial nerve net is prompted with a trained input pattern provides:

| | | | | | | | | |
|---|---|---|---|---|---|---|---|---|
| 1 | 0 | 0 | 0 | 0 | 0 | 0 | 0 | 0 |

The above state represents 25% recall of the output pattern.

Model 2: Synaptogenesis Absent Neurogenesis after Atrophy or Trauma

Absent growth of new neurons, operating under the parameters of the model, the ANN reaches a saturation point of synaptogenesis. That is, once all nodes are reconnected and operational, an optimal state for the ANN is again reached based on the limitation to the number of original nodes or the number of nodes after the simulated trauma to the network.

| | | | | | | | |
|---|---|---|---|---|---|---|---|
| 1 | 0 | 0 | 0 | 1 | 0 | 0 | 1 |
| 0 | 0 | 0 | 0 | 0 | 0 | 0 | 0 |
| 1 | 0 | 0 | 0 | 1 | 0 | 0 | 1 |
| 1 | 0 | 0 | 0 | 1 | 0 | 0 | 1 |
| 1 | 0 | 0 | 0 | 1 | 0 | 0 | 1 |
| 0 | 0 | 0 | 0 | 0 | 0 | 0 | 0 |
| 0 | 0 | 0 | 0 | 0 | 0 | 0 | 0 |
| 0 | 0 | 0 | 0 | 0 | 0 | 0 | 0 |
| 1 | 0 | 0 | 0 | 1 | 0 | 0 | 1 |

Output of a thresholding function when simulating recall of the correct output pattern when the artificial nerve net is prompted with a trained input pattern provides:

| | | | | | | | | |
|---|---|---|---|---|---|---|---|---|
| 1 | 0 | 0 | 0 | 1 | 0 | 0 | 1 | 0 |

The above state represents 75% recall of the output pattern.

Model 3: Synaptogenesis with Neurogenesis after Atrophy or Trauma

| | | | | | | | | |
|---|---|---|---|---|---|---|---|---|
| 1 | 0 | 0 | 0 | 1 | 0 | 0 | 1 | 1 |
| 0 | 0 | 0 | 0 | 0 | 0 | 0 | 0 | 0 |
| 1 | 0 | 0 | 0 | 1 | 0 | 0 | 1 | 1 |
| 1 | 0 | 0 | 0 | 1 | 0 | 0 | 1 | 1 |
| 1 | 0 | 0 | 0 | 1 | 0 | 0 | 1 | 1 |
| 0 | 0 | 0 | 0 | 0 | 0 | 0 | 0 | 0 |
| 0 | 0 | 0 | 0 | 0 | 0 | 0 | 0 | 0 |
| 0 | 0 | 0 | 0 | 0 | 0 | 0 | 0 | 0 |
| 1 | 0 | 0 | 0 | 1 | 0 | 0 | 1 | 1 |

Output of a thresholding function when simulating recall of the correct output pattern when the

artificial nerve net is prompted with a trained input pattern provides:

| | | | | | | | | |
|---|---|---|---|---|---|---|---|---|
| 1 | 0 | 0 | 0 | 1 | 0 | 0 | 1 | 1 |

The above state represents 100% recall of the output pattern.

The given simulations show a synergistic effect of synaptogenesis and neurogenesis. Therefore, compounds that promote synaptogenesis, when combined with compounds that promote neurogenesis, gain a synergistic advantage.

### Advantages

In some embodiments, a short term advantage may be gained by initial relief from ketamine, but the introduction of a serotonergic psychedelic such as psilocybin, psilocin, and/or derivative of psilocybin or psilocin, into the composition also parlays a longer term treatment without additional pain. Further, detrimental side effects of continued ketamine use are stemmed, preempted, and/or circumvented at almost no additional cost. That is, the composition allows a patient to escape additional pain and/or procedures earlier on and break free from a longer or continued treatment cycle. Such a breakout may also be more cost effective for the patient. A patient may also escape would-be side effects associated with continued treatments of ketamine, for example. In this way, a synergistic effect is gained.

In some embodiments, microdoses of psilocybin, for example, may be prepared and provided for continued treatment for some patients. Such microdoses may be tailored for a patient and provided in easy to use forms, such as prepared capsules that may be added or emptied into foods, jellies, spreads, or beverages such as coffee, tea, soda, water, etc.

In some embodiments, the present invention provides a method of using a composition comprising a serotonergic psychedelic/derivative and ketamine/derivative for increasing neural plasticity of the neuronal cell. Examples of serotonergic psychedelics include ayahuasca, dimethyltryptamine, psilocybin, lysergic acid diethylamide [LSD], and mescaline. Several compounds of the compositions described herein have demonstrated to increase neuritogenesis and/or spinogenesis both in vitro and in vivo. These changes in neuronal structure are accompanied by increased synapse numbers and function as measured by fluorescence microscopy and electrophysiology.

The compositions comprising a serotonergic psychedelic/derivative and ketamine/derivative may improve mood by increasing translation of key neurotrophic factor proteins involved in neural plasticity.

The present invention provides a method of using compositions comprising a serotonergic psychedelic/derivative and ketamine/derivative for treatment of a brain disorder. The brain disorder can be a psychiatric disorder including depression, anxiety, and/or post-traumatic stress disorder. The brain disorder can be a substance use disorder. And the brain disorder can be a neurodegenerative disorder including Alzheimer's and/or Parkinson's diseases.

### Definitions

The abbreviations used herein have their conventional meaning within the chemical and biological arts.

Where substituent groups are specified by their conventional chemical formulae, written from left to right, they equally encompass the chemically identical substituents that would result from writing the structure from right to left, e.g., —CH2O— is equivalent to —OCH2-.

“Alkyl” refers to a straight or branched, saturated, aliphatic radical having the number of carbon atoms indicated. Alkyl can include any number of carbons, such as C1-C2, C1-C₃, Ci-C4, C1-C5, Ci-C6, C1-C7, Ci-C8, C1-C9, C1-C10, C2-C3, C2-C4, C2-C5, C2-C6, C3-C4, C3-C5, C3-C6, C4-C5, C4-C6 and C5-C6. For example, C1-C6 alkyl includes, but is not limited to, methyl, ethyl, propyl, isopropyl, butyl, isobutyl, secbutyl, tert-butyl, pentyl, isopentyl, hexyl, etc. Alkyl can also refer to alkyl groups having up to 20 carbons atoms, such as, but not limited to heptyl, octyl, nonyl, decyl, etc. Alkyl groups can be substituted or unsubstituted.

“Alkylene” refers to a straight or branched, saturated, aliphatic radical having the number of carbon atoms indicated, and linking at least two other groups, i.e., a divalent hydrocarbon radical. The two moieties linked to the alkylene can be linked to the same atom or different atoms of the alkylene group. For instance, a straight chain alkylene can be the bivalent radical of —(CH₂)ₙ—, where n is 1, 2, 3, 4, 5 or 6. Representative alkylene groups include, but are not limited to, methylene, ethylene, propylene, isopropylene, butylene, isobutylene, sec-butylene, pentylene and hexylene. Alkylene groups can be substituted or unsubstituted.

“Alkenyl” refers to a straight chain or branched hydrocarbon having at least 2 carbon atoms and at least one double bond. Alkenyl can include any number of carbons, such as C2, C2-C3, C2-C4, C2-C5, C2-C6, C2-C7, C2-C8, C2-C9, C2-C10, C3, C3-C4, C3-C5, C3-C6, C4, C4-C5, C4-C5, C5, C5-C6, and C6. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. Alkenyl groups can be substituted or unsubstituted.

“Cycloalkyl” refers to a saturated or partially unsaturated, monocyclic, fused bicyclic or bridged polycyclic ring assembly containing from 3 to 12 ring atoms, or the number of atoms indicated. Cycloalkyl can include any number of carbons, such as C3-C6, C4-C6, C5-C6, C3-C8, C4-C8, C5-C8, C6-C8, C3-C9, C3-C10, C3-C11, and C3-C12. Saturated monocyclic cycloalkyl rings include, for example, cyclopropyl, cyclobutyl, cyclopentyl, cyclohexyl, and cyclooctyl.

Saturated bicyclic and polycyclic cycloalkyl rings include, for example, norbornane, [2.2.2] bicyclooctane, decahydronaphthalene and adamantane. Cycloalkyl groups can also be partially unsaturated, having one or more double or triple bonds in the ring. Representative cycloalkyl groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene. When cycloalkyl is a saturated monocyclic C3-8 cycloalkyl, exemplary groups include, but are not limited to cyclopropyl, cyclobutyl, cyclopentyl, cyclohexyl, cycloheptyl and cyclooctyl. When cycloalkyl is a saturated monocyclic C3-C8 cycloalkyl, exemplary groups include, but are not limited to cyclopropyl, cyclobutyl, cyclopentyl, and cyclohexyl. Cycloalkyl groups can be substituted or unsubstituted.

“Alkoxy” refers to an alkyl group having an oxygen atom that connects the alkyl group to the point of attachment: alkyl-O—. As for the alkyl group, alkoxy groups can have any suitable number of carbon atoms, such as Ci-C6. Alkoxy groups include, for example, methoxy, ethoxy, propoxy, iso-propoxy, butoxy, 2-butoxy, iso-butoxy, sec-butoxy, tert-butoxy, pentoxy, hexoxy, etc. The alkoxy groups can be further substituted with a variety of substituents described within. Alkoxy groups can be substituted or unsubstituted.

“Hydroxyalkyl” or “alkylhydroxy” refer to an alkyl group, as defined above, where at least one of the hydrogen atoms is replaced with a hydroxy group. As for the alkyl group, alkylhydroxy groups can have any suitable number of carbon atoms, such as C1-G5. Exemplary alkylhydroxy groups include, but are not limited to, hydroxy-methyl, hydroxyethyl (where the hydroxy is in the 1- or 2-position), hydroxypropyl (where the hydroxy is in the 1-, 2- or

3-position), hydroxybutyl (where the hydroxy is in the 1-, 2-, 3- or 4-position), hydroxypentyl (where the hydroxy is in the 1-, 2-, 3-, 4- or 5-position), hydroxyhexyl (where the hydroxy is in the 1-, 2-, 3-, 4-, 5- or 6-position), 1,2-dihydroxy ethyl, and the like.

“Halogen” refers to fluorine, chlorine, bromine and iodine.

“Haloalkyl” refers to alkyl, as defined above, where some or all of the hydrogen atoms are replaced with halogen atoms. As for the alkyl group, haloalkyl groups can have any suitable number of carbon atoms, such as Ci-C6. For example, haloalkyl includes trifluoromethyl, fluoromethyl, etc. In some instances, the term “perfluoro” can be used to define a compound or radical where all the hydrogens are replaced with fluorine. For example, perfluoromethane includes 1,1,1-trifluoromethyl.

“Amino” refers to an —N(R)2 group where the R groups can be hydrogen, alkyl, alkenyl, alkynyl, cycloalkyl, heterocycloalkyl, aryl, or heteroaryl, among others. The R groups can be the same or different. The amino groups can be primary (each R is hydrogen), or secondary (one R is hydrogen) or tertiary (each R is other than hydrogen).

“Alkylamino” refers a secondary amino group where one R is hydrogen and the other R is alkyl, as defined above. As for the alkyl group, alkylamino groups can have any suitable number of carbon atoms, such as Ci-C6. Alkylamino groups useful in the present invention include, but are not limited to, methylamino and ethylamino.

“Dialkylamino” refers a tertiary amino group where both R groups are alkyl, as defined above. As for the alkyl group, dialkylamino groups can have any suitable number of carbon atoms, such as Ci-C6. Dialkylamino groups useful in the present invention include, but are not limited to, dimethylamino and diethylamino.

“Aminoalkyl” refers to alkyl, as defined above, where one or more hydrogen atoms are replaced with an amino group. As for the alkyl group, aminoalkyl groups can have any suitable number of carbon atoms, such as Ci-C6. Aminoalkyl groups useful in the present invention include, but are not limited to, dimethylaminoethyl, dimethylaminopropyl, dimethylaminobutyl, and diethylaminopropyl.

“Heterocycloalkyl” refers to a saturated ring system having from 3 to 12 ring members and from 1 to 4 heteroatoms of N, O and S. The heteroatoms can also be oxidized, such as, but not limited to, —S(O)— and —S(O)₂—. Heterocycloalkyl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heterocycloalkyl groups, such as 1, 2, 3, or 4, or 1 to 2, 1 to 3, 1 to 4, 2 to 3, 2 to 4, or 3 to 4. The heterocycloalkyl group can include groups such as aziridine, azetidine, pyrrolidine, piperidine, azepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxalidine, thiazolidine, isothiazolidine, dioxolane, dithiolane, morpholine, thiomorpholine, dioxane, or dithiane. The heterocycloalkyl groups can also be fused to aromatic or non-aromatic ring systems to form members including, but not limited to, indoline. Heterocycloalkyl groups can be unsubstituted or substituted. For example, heterocycloalkyl groups can be substituted with

Ci-₆ alkyl or oxo (=0), among many others.

The heterocycloalkyl groups can be linked via any position on the ring. For example, aziridine can be 1- or 2-aziridine, azetidine can be 1- or 2-azetidine, pyrrolidine can be 1-, 2- or 3-pyrrolidine, piperidine can be 1-, 2-, 3- or 4-piperidine, pyrazolidine can be 1-, 2-, 3-, or 4-pyrazolidine, imidazolidine can be 1-, 2-, 3- or 4-imidazolidine, piperazine can be 1-, 2-, 3- or 4-piperazine, tetrahydrofuran can be 1- or 2-tetrahydrofuran, oxazolidine can be 2-, 3-, 4- or 5-oxazolidine, isoxazolidine can be 2-, 3-, 4- or 5-isoxazolidine, thiazolidine can be 2-, 3-, 4- or 5-thiazolidine, isothiazolidine can be 2-, 3-, 4- or 5-isothiazolidine, and morpholine can be 2-, 3- or 4-morpholine.

When heterocycloalkyl includes 3 to 8 ring members and 1 to 3 heteroatoms, representative members include, but are not limited to, pyrrolidine, piperidine, tetrahydrofuran, oxane, tetrahydrothiophene, thiane, pyrazolidine, imidazolidine, piperazine, oxazolidine, isoxazolidine, thiazolidine, isothiazolidine, morpholine, thiomorpholine, dioxane and dithiane. Heterocycloalkyl can also form a ring having 5 to 6 ring members and 1 to 2 heteroatoms, with representative members including, but not limited to, pyrrolidine, piperidine, tetrahydrofuran, tetrahydrothiophene, pyrazolidine, imidazolidine, piperazine, oxazolidine, isoxazolidine, thiazolidine, isothiazolidine, and morpholine.

“N—(Ci-C₆ alkyl)pyrrolidinyl” or “N—(Ci-C₆ alkyl)piperidinyl” refers to pyrrolidinyl or piperidinyl group, where the nitrogen (N) of the pyrrolidinyl or piperidinyl group has an alkyl group, as defined above. The pyrrolidinyl can be 1-, 2- or 3-pyrrolidinyl, piperidinyl can be 1-, 2-, 3- or 4-piperidinyl. The N—(Ci-C₆ alkyl)pyrrolidinyl groups useful in the present invention include, but are not limited to, N-methyl-2pyrrolidinyl. The N—(Ci-C6 alkyl)piperidinyl groups useful in the present invention include, but are not limited to, N-methyl-4-piperidinyl.

“Aryl” refers to an aromatic ring system having any suitable number of ring atoms and any suitable number of rings. Aryl groups can include any suitable number of ring atoms, such as, 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl. Aryl groups can be substituted or unsubstituted.

“Heteroaryl” refers to a monocyclic or fused bicyclic or tricyclic aromatic ring assembly containing 5 to 16 ring atoms, where from 1 to 5 of the ring atoms are a heteroatom such as N, O or S. The heteroatoms can also be oxidized, such as, but not limited to, —S(O)— and —S(O)₂—. Heteroaryl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heteroaryl groups, such as 1, 2, 3, 4, or 5, or 1 to 2, 1 to 3, 1 to 4, 1 to 5, 2 to 3, 2 to 4, 2 to 5, 3 to 4, or 3 to 5. Heteroaryl groups can have from 5 to 8 ring members and from 1 to 4 heteroatoms, or from 5 to 8 ring members and from 1 to 3 heteroatoms, or from 5 to 6 ring members and from 1 to 4 heteroatoms, or from 5 to 6 ring members and from 1 to 3 heteroatoms. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted.

The heteroaryl groups can be linked via any position on the ring. For example, pyrrole includes 1-, 2- and 3-pyrrole, pyridine includes 2-, 3- and 4-pyridine, imidazole includes 1-, 2-, 4- and 5-imidazole, pyrazole includes 1-, 3-, 4- and 5-pyrazole, triazole includes 1-, 4- and 5-triazole, tetrazole includes 1- and 5-tetrazole, pyrimidine includes 2-, 4-, 5- and 6-pyrimidine, pyridazine includes 3- and 4-pyridazine, 1,2,3-triazine includes 4- and 5-triazine, 1,2,4-triazine includes 3-, 5- and 6-triazine, 1,3,5-triazine includes 2-triazine, thiophene includes 2- and 3-thiophene, furan includes 2- and 3-furan, thiazole includes 2-, 4- and 5-thiazole, isothiazole includes 3-, 4- and 5-isothiazole, oxazole includes 2-, 4- and 5-oxazole, isoxazole includes 3-, 4- and 5-isoxazole, indole includes 1-, 2- and 3-indole, isoindole includes 1- and 2-isoindole, quinoline includes 2-, 3- and 4-quinoline, isoquinoline includes 1-, 3- and 4-isoquinoline, quinazoline includes 2- and 4-quinoazoline, cinnoline includes 3- and 4-cinnoline,

benzothiophene includes 2- and 3-benzothiophene, and benzofuran includes 2- and 3-benzofuran.

Some heteroaryl groups include those having from 5 to 10 ring members and from 1 to 3 ring atoms including N, O or S, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, isoxazole, indole, isoindole, quinoline, isoquinoline, quinoxaline, quinazoline, phthalazine, cinnoline, benzothiophene, and benzofuran. Other heteroaryl groups include those having from 5 to 8 ring members and from 1 to 3 heteroatoms, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, and isoxazole. Some other heteroaryl groups include those having from 9 to 12 ring members and from 1 to 3 heteroatoms, such as indole, isoindole, quinoline, isoquinoline, quinoxaline, quinazoline, phthalazine, cinnoline, benzothiophene, benzofuran and bipyridine. Still other heteroaryl groups include those having from 5 to 6 ring members and from 1 to 2 ring heteroatoms including N, O or S, such as pyrrole, pyridine, imidazole, pyrazole, pyrazine, pyrimidine, pyridazine, thiophene, furan, thiazole, isothiazole, oxazole, and isoxazole.

Some heteroaryl groups include from 5 to 10 ring members and only nitrogen heteroatoms, such as pyrrole, pyridine, imidazole, pyrazole, triazole, pyrazine, pyrimidine, pyridazine, triazine (1,2,3-, 1,2,4- and 1,3,5-isomers), indole, isoindole, quinoline, isoquinoline, quinoxaline, quinazoline, phthalazine, and cinnoline. Other heteroaryl groups include from 5 to 10 ring members and only oxygen heteroatoms, such as furan and benzofuran. Some other heteroaryl groups include from 5 to 10 ring members and only sulfur heteroatoms, such as thiophene and benzothiophene. Still other heteroaryl groups include from 5 to 10 ring members and at least two heteroatoms, such as imidazole, pyrazole, triazole, pyrazine, pyrimidine, pyridazine, triazine (1,2,3-, 1,2,4- and 1,3,5-isomers), thiazole, isothiazole, oxazole, isoxazole, quinoxaline, quinazoline, phthalazine, and cinnoline.

“Salt” refers to acid or base salts of the compounds used in the methods of the present invention. Illustrative examples of pharmaceutically acceptable salts are mineral acid

(hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

Pharmaceutically acceptable salts of the acidic compounds of the present invention are salts formed with bases, namely cationic salts such as alkali and alkaline earth metal salts, such as sodium, lithium, potassium, calcium, magnesium, as well as ammonium salts, such as ammonium, trimethyl-ammonium, diethylammonium, and tris-(hydroxymethyl)-methyl-ammonium salts.

Similarly acid addition salts, such as of mineral acids, organic carboxylic and organic sulfonic acids, e.g., hydrochloric acid, methanesulfonic acid, maleic acid, are also possible provided a basic group, such as pyridyl, constitutes part of the structure.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

“Hydrate” refers to a compound that is complexed to at least one water molecule. The compounds of the present invention can be complexed with from 1 to 10 water molecules.

“Isomers” refers to compounds with the same chemical formula but which are structurally distinguishable. Certain compounds of the present invention possess asymmetric carbon atoms (optical centers) or double bonds; the racemates, diastereomers, geometric isomers and individual isomers are all intended to be encompassed within the scope of the present invention.

“Tautomer” refers to one of two or more structural isomers which exist in equilibrium and which are readily converted from one form to another.

The present invention includes all tautomers and stereoisomers of compounds in the compositions of the present invention, either in admixture or in pure or substantially pure form. The compounds of the present invention can have asymmetric centers at the carbon atoms, and therefore the compounds of the present invention can exist in diastereomeric or enantiomeric forms or mixtures thereof. All conformational isomers (e.g., cis and trans isomers) and all optical isomers (e.g., enantiomers and diastereomers), racemic, diastereomeric and other mixtures of such isomers, as well as solvates, hydrates, isomorphs, polymorphs and tautomers are within the scope of the present invention. Compounds according to the present invention can be prepared using diastereomers, enantiomers or racemic mixtures as starting materials. Furthermore, diastereomer and enantiomer products can be separated by chromatography, fractional crystallization or other methods known to those of skill in the art.

“Sholl analysis” is a method of quantitative analysis commonly used in neuronal studies to characterize the morphological characteristics of an imaged neuron. It creates a series of concentric circles around the soma of the neuron, and counts how many times the neuron intersects with the circumference of these circles. Common analysis methods include linear analysis, semi-log analysis, and log-log analysis.

The linear method is the analysis of the function N(r), where N is the number of crossings for a circle of radius r. The critical value is the radius r at which there is a maximum number of dendritic crossings, this value is closely related to the dendrite maximum (Nmax). Dendrite maximum (Nmax) is the maximum of the function N(r), as specified by the critical value for a given data set. Schoenen Ramification Index is one measure of the branching of the neuronal cell being studied. It is calculated by dividing the dendrite maximum by the number of primary dendrites, that is, the number of dendrites originating at the cell's soma.

“A Sholl plot” refers to a plot with the number of crossings (N) at the Y axis of the plot and the radius r of the circle at the X axis of the plot. The Sholl plot provides an area-under-curve (AUC).

From an imaged neuron, other parameters can also be obtained to measure arbor complexity, for example, a number of dendritic branches, a number of primary dendrites, a total dendritic length, and a length of longest dendrite.

“A number of dendritic branches” refers to the total number of branches per neuron.

“A number of primary dendrites” refers to the number of dendrites originating at the cell's soma.

“A total dendritic length” refers to the total length of all dendrites per neurons.

“A length of longest dendrite” refers to the length of the longest dendrite for a particular neuron.

A dendritic spine (or spine) is a small membranous protrusion from a neuron's dendrite that typically receives input from a single axon at the synapse. Dendritic spines serve as a storage site for synaptic strength and help transmit electrical signals to the neuron's cell body. Most spines have a bulbous head (the spine head), and a thin neck that connects the head of the spine to the shaft of the dendrite. Dendritic spines are small with spine head volumes ranging 0.01 μm³ to 0.8 μm³. Spines with strong synaptic contacts typically have a large spine head, which connects to the dendrite via a membranous neck. The most notable classes of spine shape are “thin”, “filopodium”, “stubby”, and “mushroom”.

“A density of dendritic spines” refers to numbers of spines per 10 μm (the length of dendrite).

Synapses is defined in the present invention as a colocalization of a presynaptic protein (VGLUT1 puncta) and a post-synaptic protein (PSD-95 puncta).

“A density of synapse” refers to numbers of synapses per unit length (of the dendrite)

“Fold” refers to the fold ratio (also called fold change), and is the ratio of the measured value for an experimental sample to the measured value for the control sample. In the present invention, the measured value of a property of the neuronal cells treated with the composition comprising a serotonergic psychedelic/derivative and ketamine/derivative (i.e., the composition) is compared to the one treated with a vehicle solution without the testing compound (i.e., the vehicle control). The ratio of the measured value of the neuronal cells treated with the composition to the vehicle control is determined by fold. The property of the neuronal cells includes, but is not limited to, a maximum number of dendritic crossings, an AUC, a number of dendritic branches, a number of primary dendrites, a total dendritic length, a length of longest dendrite, a density of dendritic spines, a density of synapse, a density of a presynaptic protein (e.g., VGLUT1), a density of a postsynaptic protein (e.g., PSD-95),), a density of colocalization of presynaptic (e.g., VGLUT1) and postsynaptic (e.g., PSD-95), and translation, transcription, or secretion of neurotrophic factors (e.g., BDNF and GDNF).

“Composition” as used herein is intended to encompass a product comprising the specified ingredients in the specified amounts, as well as any product, which results, directly or indirectly, from combination of the specified ingredients in the specified amounts. By “pharmaceutically acceptable” it is meant the carrier, diluent or excipient must be compatible with the other ingredients of the formulation and deleterious to the recipient thereof.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors and colors, and the like. One of skill in the art will recognize that other pharmaceutical excipients are useful in the present invention.

“Treat”, “treating” and “treatment” refer to any indicia of success in the treatment or amelioration of an injury, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation.

“Patient” or “subject in need thereof refers to a living organism suffering from or prone to a condition that can be treated by administration of a pharmaceutical composition as provided herein. Non-limiting examples include humans, other mammals, for example primates cows, sheep, goats, horses, dogs, cats, rabbits, rats, mice and the like, and other non-mammalian animals.

“Disorder” or “condition” refer to a state of being or health status of a patient or subject capable of being treated with the composition comprising a serotonergic psychedelic/derivative and ketamine/derivative of the present invention. Examples of disorders or conditions include, but are not limited to, a psychiatric disorder such as depression, anxiety, and post-traumatic stress disorder; a substance use disorder such as addition; and a neurodegenerative disorder such as Alzheimer's and Parkinson's diseases.

“Administering” refers to oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject.

“Therapeutically effective amount or dose” or “therapeutically sufficient amount or dose” or “effective or sufficient amount or dose” refer to a dose that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). In sensitized cells, the therapeutically effective dose can often be lower than the conventional therapeutically effective dose for non-sensitized cells. [0068] Description of compounds of the present invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, or physiological conditions.

Methods of Increasing Neural Plasticity

In one aspect, provided herein is a method of increasing neural plasticity. The method includes contacting a neuronal cell with a composition comprising a serotonergic psychedelic/derivative and ketamine/derivative, in an amount sufficient to increase neural plasticity of the neuronal cell, wherein the composition produces a maximum number of dendritic crossings with an increase of greater than 1.0 fold by a Sholl Analysis.

The neuronal cells can be any type of neuron cells. In some embodiments, the neuron cell is a cortical neuron cell. In some embodiments, the neuron cell is a cortical pyramidal neuron cell.

Determining Neural Plasticity Promoted by Compositions Comprising a Serotonergic Psychedelic/Serotonergic Psychedelic Derivative and Ketamine/Ketamine Derivative

Neural plasticity refers to the ability of neurons to change in form and function in response to alterations in their environment. The neural plasticity can be evaluated by neuritogenesis, spinogenesis, and synaptogenesis in neurons. Neurogenesis is the formation of neurites. Spinogenesis is the development of dendritic spines in neurons. Synaptogenesis is the formation of synapses between neurons in the nervous system.

Neuritogenesis in Neurons

First, the composition comprising a serotonergic psychedelic/derivative and ketamine/derivative (i.e., the compounds) promotes neuritogenesis in neuronal cells. A Sholl analysis can be used to study the neuritogenesis by evaluating, for example, a maximum number of dendritic crossings and an area-under-curve (AUC) of the Sholl plot. From an imaged neuron, neuritogenesis can also be evaluated by a number of dendritic branches, a number of primary dendrites, a total dendritic length, and a length of longest dendrite.

In some embodiments, the composition comprising a serotonergic psychedelic/derivative and ketamine/derivative produces a maximum number of dendritic crossings with an increase of greater than 1.0 fold by the Sholl Analysis. In some embodiments, the composition comprising a serotonergic psychedelic/derivative and ketamine/derivative produces a maximum number of dendritic crossings with an increase of greater than 1.2 fold, greater than 1.5 fold, or greater than 2.0 fold, by the Sholl Analysis. In other embodiments, the composition comprising a serotonergic psychedelic/derivative and ketamine/derivative produces a maximum number of dendritic crossings with an increase of from 1.0 to 3.0 fold, from 1.0 to 2.5 fold, from 1.0 to 2.0 fold, from 1.0 to 1.5 fold, from 1.5 to 3.0 fold, from 1.2 to 2.5 fold, from 1.5 to 2.5 fold, from 1.2 to 2.0 fold, from 1.5 to 2.0 fold, or from 1.2 to 1.5 fold, by the Sholl Analysis.

The composition comprising a serotonergic psychedelic/derivative and ketamine/derivative also has a similar effect on other morphologies of neurons, for example, an area-under-curve (AUC) of the Sholl plot, a number of dendritic branches, and a total dendritic length.

In some embodiments, the composition comprising a serotonergic psychedelic/derivative and ketamine/derivative produces an area-under-curve (AUC) of the Sholl plot with an increase of greater than 1.0 fold by the Sholl Analysis.

In other embodiments, the composition comprising a serotonergic psychedelic/derivative and ketamine/derivative produces an AUC of the Sholl plot with an increase of greater than 1.2 fold, greater than 1.5 fold, or greater than 2.0 fold. In still other embodiments, the composition comprising a serotonergic psychedelic/derivative and ketamine/derivative produces the AUC of the Sholl plot with an increase of from 1.0 to 3.0 fold, from 1.0 to 2.5 fold, from 1.0 to 2.0 fold, from 1.0 to 1.5 fold, from 1.5 to 3.0 fold, from 1.2 to 2.5 fold, from 1.5 to 2.5 fold, from 1.2 to 2.0 fold, from 1.5 to 2.0 fold, or from 1.2 to 1.5 fold.

In some embodiments, the composition comprising a serotonergic psychedelic/derivative and ketamine/derivative produces a number of dendritic branches with an increase of greater than 1.0 fold.

In other embodiments, the composition comprising a serotonergic psychedelic/derivative and ketamine/derivative produces a number of dendritic branches with an increase of greater than 1.2 fold, greater than 1.5 fold, or greater than 2.0 fold. In still other embodiments, the composition comprising a serotonergic psychedelic/derivative and ketamine/derivative produces a number of dendritic branches with an increase of from 1.0 to 3.0 fold, from 1.0 to 2.5 fold, from 1.0 to 2.0 fold, from 1.0 to 1.5 fold, from 1.5 to 3.0 fold, from 1.2 to 2.5 fold, from 1.5 to 2.5 fold, from 1.2 to 2.0 fold, from 1.5 to 2.0 fold, or from 1.2 to 1.5 fold.

In some embodiments, the composition comprising a serotonergic psychedelic/derivative and ketamine/derivative produces a total dendritic length with an increase of greater than 1.0 fold.

In other embodiments, the composition comprising a serotonergic psychedelic/derivative and ketamine/derivative produces a total dendritic length with an increase of greater than 1.2 fold, greater than 1.5 fold, or greater than 2.0 fold. In still other embodiments, the composition comprising a serotonergic psychedelic/derivative and ketamine/derivative produces a total dendritic length with an increase of from 1.0 to 3.0 fold, from 1.0 to 2.5 fold, from 1.0 to 2.0 fold, from 1.0 to 1.5 fold, from 1.5 to 3.0 fold, from 1.2 to 2.5 fold, from 1.5 to 2.5 fold, from 1.2 to 2.0 fold, from 1.5 to 2.0 fold, or from 1.2 to 1.5 fold.

The composition comprising a serotonergic psychedelic/derivative and ketamine/derivative may have a limited effect on a number of primary dendrites. In some embodiments, the composition comprising a serotonergic psychedelic/derivative and ketamine/derivative produces a number of primary dendrites with an increase of greater than 1.0 fold. In other embodiments, the composition comprising a serotonergic psychedelic/derivative and ketamine/derivative produces a number of primary dendrites at about 1.0 fold.

The composition comprising a serotonergic psychedelic/derivative and ketamine/derivative may have a limited effect on a length of the longest dendrite. In some embodiments, the composition comprising a serotonergic psychedelic/derivative and ketamine/derivative produces a length of the longest dendrite with an increase of greater than 1.0 fold. In other embodiments, the composition comprising a serotonergic psychedelic/derivative and ketamine/derivative produces a length of the longest dendrite at about 1.0 fold.

The neuronal cells can be treated with various concentrations of the composition comprising a serotonergic psychedelic/derivative and ketamine/derivative. In some embodiments, the neuronal cells are treated with the composition comprising a serotonergic psychedelic/derivative and ketamine/derivative at a concentration of 90 μM, 10 μM, 100 nM, 1 nM, 10 pM, or 0.1 pM. In one specific embodiment, the neuronal cells are treated with the composition at a concentration of 90 μM. In another specific embodiment, the neuronal cells are treated with the composition at a concentration of 10 μM.

In some embodiments, a composition comprises a psychedelic compound comprising a serotonergic psychedelic compound and ketamine. In some embodiments, the psychedelic compound, drug, or pharmaceutical is chosen from a tryptamine, phenethylamine, or lysergamide. In some embodiments, the psychedelic compound, drug, or pharmaceutical is chosen from psilocybin, psilocin, or a psilocybin derivative. In some embodiments, the ketamine is S-ketamine. In some embodiments, the ketamine is S-ketamine hydrochloride.

In some embodiments, the concentration of the ketamine is at least 110 mg/mL of total composition volume. In some embodiments, the concentration of the ketamine is at least 125 mg/mL, based on the total volume of the composition. In some embodiments, the concentration of the ketamine is at least 130 mg/mL, based on the total volume of the composition. In some embodiments, ketamine is present in a concentration in the range of eq. 125 mg/mL to eq. 250 mg/mL, based on the total volume of the composition. In some embodiments, ketamine is present in a concentration in the range of eq. 125 mg/mL to eq. 180 mg/mL, based on the total volume of the composition. In some embodiments, ketamine is present in a concentration in the range of eq. 125 mg/mL to eq. 150 mg/mL, based on the total volume of the composition. In some embodiments, ketamine is present in a concentration in the range of eq. 126 mg/mL to eq. 162 mg/mL, based on the total volume of the composition. In some embodiments, the ketamine is present at a concentration in the range of 125 mg/mL equivalents to 200 mg/mL equivalents based on the total volume of the composition; or the ketamine is present at a concentration in the range of 125 mg/mL equivalents to 180 mg/mL equivalents based on the total volume of the composition; or wherein the ketamine is present at a concentration in the range of 125 mg/mL equivalents to 150 mg/mL equivalents based on the total volume of the composition.

In some embodiments, the composition has a pH value within a range from 3.5 to 6.5. In some embodiments, the composition has a pH value within a range from 4.0 to 6.5. In some embodiments, the composition has a pH value within a range from 4.0 to 5.5. In some embodiments, the composition has a pH value within a range from 3.5 to 5.5.

In some embodiments, the psychedelic compound, drug, or pharmaceutical and the ketamine are present in the composition in a molar ratio of between 100:1 to 1:100. In some embodiments, the molar ratio is between 75:1 to 1:75. In some embodiments, the molar ratio is between 50:1 to 1:50. In some embodiments, the molar ratio is between 25:1 to 1:25. In some embodiments, the molar ratio is between 10:1 to 1:10. In some embodiments, the molar ratio is between 5:1 to 1:5. In some embodiments, the molar ratio is between 1.5:1 to 1:1.5. In some embodiments, the molar ratio is between 1.1:1 to 1:1.1.

In some embodiments, a composition comprising a serotonergic psychedelic/derivative and ketamine/derivative at a concentration of 10 μM produces a maximum number of dendritic crossings with an increase of greater than 1.0 fold by the Sholl Analysis. In some embodiments, the composition comprising a serotonergic psychedelic/derivative and ketamine/derivative at a concentration of 100 nM produces a maximum number of dendritic crossings with an increase of greater than 1.0 fold by the Sholl Analysis. In other embodiments, the composition comprising a serotonergic psychedelic/derivative and ketamine/derivative at a concentration of 1 nM produces a maximum number of dendritic crossings with an increase of greater than 1.0 fold by the Sholl Analysis. In other embodiments, the composition comprising a serotonergic psychedelic/derivative and ketamine/derivative at a concentration of 10 pM produces a maximum number of dendritic crossings with an increase of greater than 1.0 fold by the Sholl Analysis. In still other embodiments, the composition comprising a serotonergic psychedelic/derivative and ketamine/derivative at a concentration of 0.1 pM produces a maximum number of dendritic crossings with an increase of greater than 1.0 fold by the Sholl Analysis.

In some embodiments, the composition comprising a serotonergic psychedelic/derivative and ketamine/derivative at a concentration of 10 μM produces an AUC of the Sholl plot with an increase of greater than 1.0 fold by the Sholl Analysis. In some embodiments, the composition comprising a serotonergic psychedelic/derivative and ketamine/derivative at a concentration of 100 nM produces an AUC of the Sholl plot with an increase of greater than 1.0 fold by the Sholl Analysis. In other embodiments, the composition comprising a serotonergic psychedelic/derivative and ketamine/derivative at a concentration of 1 nM produces an AUC of the Sholl plot with an increase of greater than 1.0 fold by the Sholl Analysis. In other embodiments, the composition comprising a serotonergic psychedelic/derivative and ketamine/derivative at a concentration of 10 pM produces an AUC of the Sholl plot with an increase of greater than 1.0 fold by the Sholl Analysis. In still other embodiments, the composition comprising a serotonergic psychedelic/derivative and ketamine/derivative at a concentration of 0.1 pM produces an AUC of the Sholl plot with an increase of greater than 1.0 fold by the Sholl Analysis.

In some embodiments, the composition comprising a serotonergic psychedelic/derivative and ketamine/derivative at a concentration of 10 μM produces a number of dendritic branches with an increase of greater than 1.0 fold. In some embodiments, the composition comprising a serotonergic psychedelic/derivative and ketamine/derivative at a concentration of 100 nM produces a number of dendritic branches with an increase of greater than 1.0 fold. In other embodiments, the composition comprising a serotonergic psychedelic/derivative and ketamine/derivative at a concentration of 1 nM produces a number of dendritic branches with an increase of greater than 1.0 fold. In other embodiments, the composition comprising a serotonergic psychedelic/derivative and ketamine/derivative at a concentration of 10 pM produces a number of dendritic branches with an increase of greater than 1.0 fold. In still other embodiments, the composition comprising a serotonergic psychedelic/derivative and ketamine/derivative at a concentration of 0.1 pM produces a number of dendritic branches with an increase of greater than 1.0 fold.

In some embodiments, the composition comprising a serotonergic psychedelic/derivative and ketamine/derivative at a concentration of 10 μM produces a total dendritic length with an increase of greater than 1.0 fold. In some embodiments, the composition comprising a serotonergic psychedelic/derivative and ketamine/derivative at a concentration of 100 nM produces a total dendritic length with an increase of greater than 1.0 fold. In other embodiments, the composition comprising a serotonergic psychedelic/derivative and ketamine/derivative at a concentration of 1 nM produces a total dendritic length with an increase of greater than 1.0 fold. In other embodiments, the composition comprising a serotonergic psychedelic/derivative and ketamine/derivative at a concentration of 10 pM produces a total dendritic length with an increase of greater than 1.0 fold. In still other embodiments, the composition comprising a serotonergic psychedelic/derivative and ketamine/derivative at a concentration of 0.1 pM produces a total dendritic length with an increase of greater than 1.0 fold.

The neurogenesis, promoted by the composition comprising a serotonergic psychedelic/derivative and ketamine/derivative, can be studied in vitro with neuronal cells as well as in vivo or ex vivo. For Example, Drosophila larvae during various instars or zebrafish embryos can be treated with the composition to assess the in vivo effects of the compound on neurogenesis.

Spinogenesis in Neurons

Second, the composition comprising a serotonergic psychedelic/derivative and ketamine/derivative promotes spinogenesis in neuronal cells. The spinogenesis can be evaluated by a density of dendritic spines, and further by a shift in spine morphology.

In some embodiments, the composition comprising a serotonergic psychedelic/derivative and ketamine/derivative produces a density of dendritic spines with an increase of greater than 1.0 fold.

In other embodiments, the composition comprising a serotonergic psychedelic/derivative and ketamine/derivative produces a density of dendritic spines with an increase of greater than 1.2 fold, greater than 1.5 fold, or greater than 2.0 fold. In still other embodiments, the composition comprising a serotonergic psychedelic/derivative and ketamine/derivative produces a density of dendritic spines with an increase of from 1.0 to 3.0 fold, from 1.0 to 2.5 fold, from 1.0 to 2.0 fold, from 1.0 to 1.5 fold, from 1.5 to 3.0 fold, from 1.2 to 2.5 fold, from 1.5 to 2.5 fold, from 1.2 to 2.0 fold, from 1.5 to 2.0 fold, or from 1.2 to 1.5 fold.

Apart from the increase of the density of dendritic spines, the composition comprising a serotonergic psychedelic/derivative and ketamine/derivative may also cause a shift in spine morphology, favoring immature (thin and filopodium) over more mature (mushroom) spine types.

The neuronal cells can be treated with various concentrations of the composition comprising a serotonergic psychedelic/derivative and ketamine/derivative, as detailed above.

In some embodiments, the composition comprising a serotonergic psychedelic/derivative and ketamine/derivative at a concentration of 10 μM produces a density of dendritic spines with an increase of greater than 1.0 fold. In some embodiments, the composition comprising a serotonergic psychedelic/derivative and ketamine/derivative at a concentration of 100 nM produces a density of dendritic spines with an increase of greater than 1.0 fold. In other embodiments, the composition comprising a serotonergic psychedelic/derivative and ketamine/derivative at a concentration of 1 nM produces a density of dendritic spines with an increase of greater than 1.0 fold. In other embodiments, the composition comprising a serotonergic psychedelic/derivative and ketamine/derivative at a concentration of 10 pM produces a density of dendritic spines with an increase of greater than 1.0 fold. In still other embodiments, the composition comprising a serotonergic psychedelic/derivative and ketamine/derivative at a concentration of 0.1 pM produces a density of dendritic spines with an increase of greater than 1.0 fold.

The spinogenesis, promoted by the composition comprising a serotonergic psychedelic/derivative and ketamine/derivative, can be studied in vitro with neuronal cells as well as in vivo or ex vivo. For Example, the effects of the compound on spinogenesis in the mPFC of adult rats using Golgi-Cox staining can be accessed.

It is also noted in in vivo studies that the composition comprising a serotonergic psychedelic/derivative and ketamine/derivative promotes spinogenesis in pyramidal neurons with an increased density of dendritic spines of apical and/or basal dendrites. In some embodiments, the composition comprising a serotonergic psychedelic/derivative and ketamine/derivative produces a density of dendritic spines of apical dendrites with an increase of greater than 1.0 fold. In some embodiments, the composition comprising a serotonergic psychedelic/derivative and ketamine/derivative produces a density of dendritic spines of basal dendrites with an increase of greater than 1.0 fold. In some other embodiments, the composition comprising a serotonergic psychedelic/derivative and ketamine/derivative produces a density of dendritic spines of both apical dendrites and basal dendrites with an increase of greater than 1.0 fold.

In addition to the structural changes in pyramidal neurons as described above, both the frequency and amplitude of spontaneous excitatory postsynaptic currents (EPSCs) can increase following the treatment with the composition.

Synaptogenesis in Neurons

Third, the composition comprising a serotonergic psychedelic/derivative and ketamine/derivative promotes synaptogenesis in neuronal cells. The spinogenesis can be evaluated by a density of synapses (i.e., number of synapses per neuron) as well as the size of synapses.

In some embodiments, the composition comprising a serotonergic psychedelic/derivative and ketamine/derivative produces a density of synapses with an increase of greater than 1.0 fold.

In other embodiments, the composition comprising a serotonergic psychedelic/derivative and ketamine/derivative produces a density of synapses with an increase of greater than 1.2 fold, greater than 1.5 fold, or greater than 2.0 fold. In still other embodiments, the composition comprising a serotonergic psychedelic/derivative and ketamine/derivative produces a density of synapse with an increase of from 1.0 to 3.0 fold, from 1.0 to 2.5 fold, from 1.0 to 2.0 fold, from 1.0 to 1.5 fold, from 1.5 to 3.0 fold, from 1.2 to 2.5 fold, from 1.5 to 2.5 fold, from 1.2 to 2.0 fold, from 1.5 to 2.0 fold, or from 1.2 to 1.5 fold.

The composition comprising a serotonergic psychedelic/derivative and ketamine/derivative may have a limited effect on the size of synapses. In some embodiments, the composition comprising a serotonergic psychedelic/derivative and ketamine/derivative produces a size of synapses at about 1.0 fold.

The increase of the density of synapses can lead to an increase of a density of a presynaptic protein such as vesicular glutamate transporter 1 (VGLUT1).

In some embodiments, the composition comprising a serotonergic psychedelic/derivative and ketamine/derivative produces a density of a presynaptic protein with an increase of greater than 1.0 fold. In other embodiments, the composition comprising a serotonergic psychedelic/derivative and ketamine/derivative produces a density of a presynaptic protein with an increase of greater than 1.2 fold, greater than 1.5 fold, or greater than 2.0 fold. In still other embodiments, the composition comprising a serotonergic psychedelic/derivative and ketamine/derivative produces a density of a presynaptic protein with an increase of from 1.0 to 3.0 fold, from 1.0 to 2.5 fold, from 1.0 to 2.0 fold, from 1.0 to 1.5 fold, from 1.5 to 3.0 fold, from 1.2 to 2.5 fold, from 1.5 to 2.5 fold, from 1.2 to 2.0 fold, from 1.5 to 2.0 fold, or from 1.2 to 1.5 fold. The presynaptic protein is vesicular glutamate transporter 1 (VGLUT1).

The increase of the density of synapses may have a limited effect on a density of a postsynaptic protein such as postsynaptic density protein 95 (PSD-95). In some embodiments, the composition comprising a serotonergic psychedelic/derivative and ketamine/derivative produces a density of a postsynaptic protein (PSD-95) at about 1.0 fold.

The neuronal cells can be treated with various concentrations of the composition comprising a serotonergic psychedelic/derivative and ketamine/derivative, as detailed above.

In some embodiments, the composition comprising a serotonergic psychedelic/derivative and ketamine/derivative at a concentration of 10 μM produces a density of synapses with an increase of greater than 1.0 fold. In some embodiments, the composition comprising a serotonergic psychedelic/derivative and ketamine/derivative at a concentration of 100 nM produces a density of synapses with an increase of greater than 1.0 fold. In other embodiments, the composition comprising a serotonergic psychedelic/derivative and ketamine/derivative at a concentration of 1 nM produces a density of synapses with an increase of greater than 1.0 fold. In other embodiments, the composition comprising a serotonergic psychedelic/derivative and ketamine/derivative at a concentration of 10 pM produces a density of synapses with an increase of greater than 1.0 fold. In still other embodiments, the composition comprising a serotonergic psychedelic/derivative and ketamine/derivative at a concentration of 0.1 pM produces a density of synapses with an increase of greater than 1.0 fold.

Translation, Transcription, and Secretion of Neurotrophic Factors

The role of brain-derived neurotrophic factor (BDNF) in both neurogenesis and spinogenesis is well-known. See Cohen-Cory, et. al., Dev. Neurobiol 2010, 70, 271-288. The composition comprising a serotonergic psychedelic/derivative and ketamine/derivative is evaluated for its effect on translation, transcription, or secretion of the neurotrophic factors in neuronal cells.

In some embodiments, the composition comprising a serotonergic psychedelic/derivative and ketamine/derivative increases at least one of translation, transcription, and secretion of neurotrophic factors. The neurotrophic factor is at least one of a brain-derived neurotrophic factor (BDNF) and a glial cell line-derived neurotrophic factor (GDNF). In some embodiments, the neurotrophic factor is BDNF.

The composition comprising a serotonergic psychedelic/derivative and ketamine/derivative increases translation of the brain-derived neurotrophic factor (BDNF). In some embodiments, the translation of the brain-derived neurotrophic factor (BDNF) has an increase of greater than 1.0 fold. In other embodiments, the translation of the brain-derived neurotrophic factor (BDNF) has an increase of greater than 1.2 fold, greater than 1.5 fold, or greater than 2.0 fold. In still other embodiments, the translation of the brain-derived neurotrophic factor (BDNF) has an increase of from 1.0 to 3.0 fold, from 1.0 to 2.5 fold, from 1.0 to 2.0 fold, from 1.0 to 1.5 fold, from 1.5 to 3.0 fold, from 1.2 to 2.5 fold, from 1.5 to 2.5 fold, from 1.2 to 2.0 fold, from 1.5 to 2.0 fold, or from 1.2 to 1.5 fold.

The composition comprising a serotonergic psychedelic/derivative and ketamine/derivative increases translation of the glial cell line-derived neurotrophic factor (GDNF). In some embodiments, the translation of the glial cell line-derived neurotrophic factor (GDNF) has an increase of greater than 1.0 fold. In other embodiments, the translation of the glial cell line-derived neurotrophic factor (GDNF) has an increase of greater than 1.2 fold, greater than 1.5 fold, or greater than 2.0 fold. In still other embodiments, the translation of the glial cell line-derived neurotrophic factor (GDNF) has an increase of from 1.0 to 3.0 fold, from 1.0 to 2.5 fold, from 1.0 to 2.0 fold, from 1.0 to 1.5 fold, from 1.5 to 3.0 fold, from 1.2 to 2.5 fold, from 1.5 to 2.5 fold, from 1.2 to 2.0 fold, from 1.5 to 2.0 fold, or from 1.2 to 1.5 fold.

The composition comprising a serotonergic psychedelic/derivative and ketamine/derivative may have a minimal effect on transcription of the neurotrophic factors in neuronal cells. Therefore, the composition may not impact the neurotrophic factors at the level of gene expression.

Compositions Comprising a Serotonergic Psychedelic/Derivative and Ketamine/Derivative

Neurotrophic factors are known to promote neuronal survival, neurogenesis, and neural plasticity and have been studied for treating neuropsychiatric and neurodegenerative disorders. However, these large water-soluble proteins do not readily cross the blood-brain barrier (BBB).

The compounds of compositions comprising a serotonergic psychedelic/derivative and ketamine/derivative can have optimized physical properties giving them direct access to the brain. These compounds can increase endogenous brain levels of neurotrophic factors, thereby promoting neural plasticity in a brain region. Accordingly, in some embodiments, the one or more of the compounds of the composition comprising a serotonergic psychedelic/derivative and ketamine/derivative is a blood-brain-barrier (BBB) penetrator.

Without being bound to a particular theory, the composition comprising a serotonergic psychedelic/derivative and ketamine/derivative may have a novel “inside-out” mechanism by interacting with Sigma-1 receptors for their contribution to the beneficial effects of the compounds.

The one or more of the compounds in the compositions comprising a serotonergic psychedelic/derivative and ketamine/derivative can be weak bases. As weak bases with pKa's of their conjugate acids falling within a narrow range of 7-10, these compounds can exist in both protonated and deprotonated states at physiological pH. Accordingly, in some embodiments, the composition comprising a serotonergic psychedelic/derivative and ketamine/derivative has a pKa of from 7.0 to 10.0.

In the deprotonated state, these compounds can readily cross cell membranes. However, their basicity causes them to accumulate in the acidic compartments of the secretory pathway. There, they can reach high local concentrations, bind to receptors (e.g., Sigma-1 receptors), and elicit effects. Accordingly, in some embodiments, the composition comprising a serotonergic psychedelic/derivative and ketamine/derivative is permeable across cell membranes.

The compounds of the present invention can also be the salts and isomers thereof. In some embodiments, the compounds of the present invention include the salt forms thereof.

Examples of applicable salt forms include hydrochlorides, hydrobromides, sulfates,

methanesulfonates, nitrates, maleates, acetates, citrates, fumarates, tartrates (e.g. (+)-tartrates, (−)-tartrates or mixtures thereof including racemic mixtures), succinates, benzoates and salts with amino acids such as glutamic acid. These salts may be prepared by methods known to those skilled in art. When compounds of the present invention contain relatively basic functionalities, acid addition salts can be obtained by contacting the neutral form of such compounds with a sufficient amount of the desired acid, either neat or in a suitable inert solvent. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolyl sulfonic, citric, tartaric, methanesulfonic, and the like. Also included are salts of amino acids such as arginate and the like, and salts of organic acids like glucuronic or galactunoric acids and the like (see, for example, Berge et al, “Pharmaceutical Salts”, Journal of Pharmaceutical Science, 1977, 66, 1-19). Certain specific compounds of the present invention contain basic acidic functionalities that allow the compounds to be converted into base addition salts. Additional information on suitable pharmaceutically acceptable salts can be found in Remington's Pharmaceutical Sciences, 17th ed., Mack Publishing Company, Easton, Pa., 1985, which is incorporated herein by reference.

The neutral forms of the compounds are preferably regenerated by contacting the salt with a base or acid and isolating the parent compound in the conventional manner. The parent form of the compound differs from the various salt forms in certain physical properties, such as solubility in polar solvents.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques.

Isomers include compounds having the same number and kind of atoms, and hence the same molecular weight, but differing in respect to the structural arrangement or configuration of the atoms.

It will be apparent to one skilled in the art that certain compounds of this invention may exist in tautomeric forms, all such tautomeric forms of the compounds being within the scope of the invention. Tautomer refers to one of two or more structural isomers which exist in equilibrium and which are readily converted from one isomeric form to another.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, the compounds of the present invention may also contain unnatural proportions of atomic isotopes at one or more of the atoms that constitute such compounds. For example, the compounds of the present invention may be radiolabeled with radioactive isotopes, such as for example deuterium (2H), tritium (3H), iodine-125 (125I), carbon-13 (13C), or carbon-14 (14C). All isotopic variations of the compounds of the present invention, whether radioactive or not, are encompassed within the scope of the present invention.

In addition to salt forms, the present invention provides compounds, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

The compounds of the invention can be synthesized by a variety of methods known to one of skill in the art (see Comprehensive Organic Transformations Richard C. Larock, 1989) or by an appropriate combination of generally well known synthetic methods. Techniques useful in synthesizing the compounds of the invention are both readily apparent and accessible to those of skill in the relevant art. The discussion below is offered to illustrate certain of the diverse methods available for use in assembling the compounds of the invention. However, the discussion is not intended to define the scope of reactions or reaction sequences that are useful in preparing the compounds of the present invention. One of skill in the art will appreciate that other methods of making the compounds are useful in the present invention. Although some compounds described may indicate relative stereochemistry, the compounds may exist as a racemic mixture or as either enantiomer.

The composition comprising a serotonergic psychedelic/derivative and ketamine/derivative can also be evaluated using a drug discrimination behavior model. This animal behavior model is described in detail in Appel et. al. Neuroscience & Biobehavioral Reviews, Vol 6, pp. 529-536, 1982.

The composition comprising a serotonergic psychedelic/derivative and ketamine/derivative may be evaluated by in vitro methods.

In some embodiments, the present invention provides a pharmaceutical composition including a pharmaceutically acceptable excipient and a composition comprising a serotonergic psychedelic/derivative and ketamine/derivative of the present invention.

In some embodiments of the pharmaceutical compositions, the composition comprising a serotonergic psychedelic/derivative and ketamine/derivative, or a pharmaceutically acceptable salt thereof, is included in a therapeutically effective amount.

In some embodiments of the pharmaceutical compositions, the composition comprising a serotonergic psychedelic/derivative and ketamine/derivative is a compound of Formula 1a or 1b, or a pharmaceutically salt thereof. In some embodiments of the pharmaceutical compositions, the composition comprising a serotonergic psychedelic/derivative and ketamine/derivative is a compound of Formula 1a, or a pharmaceutically salt thereof. In some embodiments of the pharmaceutical compositions, the composition comprising a serotonergic psychedelic/derivative and ketamine/derivative is a compound of Formula 1b, or a pharmaceutically salt thereof.

In some embodiments of the pharmaceutical compositions, the composition comprising a serotonergic psychedelic/derivative and ketamine/derivative is a compound of Formula Ila or lib, or a pharmaceutically salt thereof. In some embodiments of the pharmaceutical compositions, the composition comprising a serotonergic psychedelic/derivative and ketamine/derivative is a compound of Formula Ila, or a pharmaceutically salt thereof. In some embodiments of the pharmaceutical compositions, the composition comprising a serotonergic psychedelic/derivative and ketamine/derivative is a compound of Formula lib, or a pharmaceutically salt thereof.

In some embodiments of the pharmaceutical compositions, the composition comprising a serotonergic psychedelic/derivative and ketamine/derivative includes Ergometrine, Dihydroergotamine, Methylergometrine, Methysergide, Ergotamine, Cabergoline, Pergolide, Lisuride, 2-Bromo-lysergic acid diethylamide (BOL-148), Nicergoline, Bromocriptine, or combinations thereof.

In other embodiments of the pharmaceutical compositions, the composition comprising a serotonergic psychedelic/derivative and ketamine/derivative includes Sumatriptan, Zolmitriptan, Rizatriptan, Eletriptan, Naratriptan, Frovatriptan, Almotriptan, 6-methoxy-N,N-dimethyltryptamine, 6-fluoro-N,N-dimethyltryptamine, or combination thereof.

In some embodiments of the pharmaceutical compositions, the pharmaceutical composition includes a second agent (e.g. therapeutic agent). In some embodiments of the pharmaceutical compositions, the pharmaceutical composition includes a second agent (e.g. therapeutic agent) in a therapeutically effective amount. In some embodiments of the pharmaceutical compositions, the second agent is an agent for treating a brain disorder. In some embodiments, the second agent is an anti-psychiatric disorder agent. In other embodiments, the second agent is an anti-substance use disorder agent. In other embodiments, the second agent is an anti-neurodegenerative agent.

Formulations

The compositions of the present invention can be prepared in a wide variety of oral, parenteral and topical dosage forms. Oral preparations include tablets, pills, powder, dragees, capsules, liquids, lozenges, cachets, gels, syrups, slurries, suspensions, etc., suitable for ingestion by the patient. The compositions of the present invention can also be administered by injection, that is, intravenously, intramuscularly, intracutaneously, subcutaneously, intraduodenally, or intraperitoneally. Also, the compositions described herein can be administered by inhalation, for example, intranasally. Additionally, the compositions of the present invention can be administered transdermally. The compositions of this invention can also be administered by intraocular, intravaginal, and intrarectal routes including suppositories, insufflation, powders and aerosol formulations (for examples of steroid inhalants, see Rohatagi, J. Clin. Pharmacol.

35: 1187-1193, 1995; Tjwa, Ann. Allergy Asthma Immunol. 75: 107-111, 1995). Accordingly, the present invention also provides pharmaceutical compositions including a pharmaceutically acceptable carrier or excipient and a compound of the present invention.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton Pa. (“Remington's”).

In powders, the carrier is a finely divided solid, which is in a mixture with the finely divided active component. In tablets, the active component is mixed with the carrier having the necessary binding properties in suitable proportions and compacted in the shape and size desired. The powders and tablets preferably contain from 5% or 10% to 70% of the compounds of the present invention.

Suitable solid excipients include, but are not limited to, magnesium carbonate;

magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose,

hydroxypropylmethyl-cellulose, or sodium carboxymethylcellulose; and gums including arabic and tragacanth; as well as proteins including, but not limited to, gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, for example, push-fit capsules made of gelatin, as well as soft, sealed capsules made of gelatin and a coating such as glycerol or sorbitol. Push-fit capsules can contain the compounds of the present invention mixed with a filler or binders such as lactose or starches, lubricants such as talc or magnesium stearate, and, optionally, stabilizers. In soft capsules, the compounds of the present invention may be dissolved or suspended in suitable liquids, such as fatty oils, liquid paraffin, or liquid

polyethylene glycol with or without stabilizers.

For preparing suppositories, a low melting wax, such as a mixture of fatty acid glycerides or cocoa butter, is first melted and the compounds of the present invention are dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the compounds of the present invention in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the compounds of the present invention in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The compositions of the present invention can also be delivered as microspheres for slow release in the body. For example, microspheres can be formulated for administration via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

In another embodiment, the compositions of the present invention can be formulated for parenteral administration, such as intravenous (IV) administration or administration into a body cavity or lumen of an organ. The formulations for administration will commonly comprise a solution of the compositions of the present invention dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compositions of the present invention in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In another embodiment, the formulations of the compositions of the present invention can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hasp. Pharm. 46: 1576-1587, 1989).

Lipid-based drug delivery systems include lipid solutions, lipid emulsions, lipid dispersions, self-emulsifying drug delivery systems (SEDDS) and self-microemulsifying drug delivery systems (SMEDDS). In particular, SEDDS and SMEDDS are isotropic mixtures of lipids, surfactants and co-surfactants that can disperse spontaneously in aqueous media and form fine emulsions (SEDDS) or microemulsions (SMEDDS). Lipids useful in the formulations of the present invention include any natural or synthetic lipids including, but not limited to, sesame seed oil, olive oil, castor oil, peanut oil, fatty acid esters, glycerol esters, Labrafil®, Labrasol®, Cremophor®, Solutol®, Tween®, Capryol®, Capmul®, Captex®, and Peceol®. VI. Administration

The compounds and compositions of the present invention can be delivered by any suitable means, including oral, parenteral and topical methods. Transdermal administration methods, by a topical route, can be formulated as applicator sticks, solutions, suspensions, emulsions, gels, creams, ointments, pastes, jellies, paints, powders, and aerosols.

The pharmaceutical preparation is preferably in unit dosage form. In such form the preparation is subdivided into unit doses containing appropriate quantities of the compounds and compositions of the present invention. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The compounds and compositions of the present invention, and any other agents, can be present in any suitable amount, and can depend on various factors including, but not limited to, weight and age of the subject, state of the disease, etc. Suitable dosage ranges include from about 0.1 mg to about 10,000 mg, or about 1 mg to about 1000 mg, or about 10 mg to about 750 mg, or about 25 mg to about 500 mg, or about 50 mg to about 250 mg. Suitable dosages also include about 1 mg, 5, 10, 20, 30, 40, 50, 60, 70, 80, 90, 100, 200, 300, 400, 500, 600, 700, 800, 900 or 1000 mg.

The compounds of the present invention can be administered at any suitable frequency, interval and duration. For example, the compound of the present invention can be administered once an hour, or two, three or more times an hour, once a day, or two, three, or more times per day, or once every 2, 3, 4, 5, 6, or 7 days, so as to provide the preferred dosage level. When the compound of the present invention is administered more than once a day, representative intervals include 5, 10, 15, 20, 30, 45 and 60 minutes, as well as 1, 2, 4, 6, 8, 10, 12, 16, 20, and 24 hours. The compound of the present invention can be administered once, twice, or three or more times, for an hour, for 1 to 6 hours, for 1 to 12 hours, for 1 to 24 hours, for 6 to 12 hours, for 12 to 24 hours, for a single day, for 1 to 7 days, for a single week, for 1 to 4 weeks, for a month, for 1 to 12 months, for a year or more, or even indefinitely.

The composition can also contain other compatible therapeutic agents. The compounds described herein can be used in combination with one another, with other active agents known to be useful in increasing neural plasticity, or with adjunctive agents that may not be effective alone, but may contribute to the efficacy of the active agent.

The compounds of the present invention can be co-administered with another active agent. Co-administration includes administering the compound of the present invention and active agent within 0.5, 1, 2, 4, 6, 8, 10, 12, 16, 20, or 24 hours of each other. Co-administration also includes administering the compound of the present invention and active agent simultaneously, approximately simultaneously (e.g., within about 1, 5, 10, 15, 20, or 30 minutes of each other), or sequentially in any order. Moreover, the compound of the present invention and the active agent can each be administered once a day, or two, three, or more times per day so as to provide the preferred dosage level per day.

In some embodiments, co-administration can be accomplished by co-formulation, i.e., preparing a single pharmaceutical composition including both the compound of the present invention and the active agent. In other embodiments, the compound of the present invention and the active agent can be formulated separately.

The compound of the present invention and the active agent can be present in the compositions of the present invention in any suitable weight ratio, such as from about 1:100 to about 100:1 (w/w), or about 1:50 to about 50:1, or about 1:25 to about 25:1, or about 1:10 to about 10:1, or about 1:5 to about 5:1 (w/w). The compound of the present invention and the other active agent can be present in any suitable weight ratio, such as about 1:100 (w/w), 1:50, 1:25, 1:10, 1:5, 1:4, 1:3, 1:2, 1:1, 2:1, 3:1, 4:1, 5:1, 10:1, 25:1, 50:1 or 100:1 (w/w). Other dosages and dosage ratios of the compound of the present invention and the active agent are suitable in the compositions and methods of the present invention.

Methods of Treating a Disorder

In one aspect, provided herein is a method of treating a brain disorder. The method includes administering to a subject in need thereof a therapeutically effective amount of a composition, thereby treating the brain disorder, wherein the composition comprising a serotonergic psychedelic/derivative and ketamine/derivative increases neural plasticity of the neuronal cell; provided that the subject is not already being treated with one or more of the following:

Ergometrine for postpartum hemorrhage and postabortion hemorrhage due to uterine atony;

Dihydroergotamine for migraines or cluster headaches;

Methylergometrine for routine management after delivery of the placenta, postpartum atony and hemorrhage, subinvolution, or migraines;

Methysergide for migraines or cluster headaches;

Ergotamine for migraines or cluster headaches;

Cabergoline for hyperprolactinemic disorders or Parkinson's disease;

Pergolide for Parkinson's disease;

Lisuride for Parkinson's disease;

Nicergoline for senile dementia or other disorders with vascular origins;

Bromocriptine for pituitary tumors, Parkinson's disease, hyperprolactinaemia, neuroleptic malignant syndrome, or type 2 diabetes; or

Sumatriptan, Zolmitriptan, Rizatriptan, Eletriptan, Naratriptan, Frovatriptan, or Almotriptan for migraines or cluster headaches.

In some aspects, the method includes administering to a subject in need thereof a therapeutically effective amount of a composition comprising a serotonergic psychedelic/derivative and ketamine/derivative, thereby treating the brain disorder, wherein the composition comprising a serotonergic psychedelic/derivative and ketamine/derivative increases neural plasticity of the neuronal cell.

In other aspects, the method includes administering to a subject in need thereof a therapeutically effective amount of a composition comprising a serotonergic psychedelic/derivative and ketamine/derivative, thereby treating the brain disorder, wherein the composition comprising a serotonergic psychedelic/derivative and ketamine/derivative increases neural plasticity of the neuronal cell; provided that the subject is not already being treated with all of the following:

Ergometrine for postpartum hemorrhage and postabortion hemorrhage due to uterine atony;

Dihydroergotamine for migraines or cluster headaches;

Methylergometrine for routine management after delivery of the placenta, postpartum atony and hemorrhage, subinvolution, or migraines;

Methysergide for migraines or cluster headaches;

Ergotamine for migraines or cluster headaches;

Cabergoline for hyperprolactinemic disorders or Parkinson's disease;

Pergolide for Parkinson's disease;

Lisuride for Parkinson's disease;

Nicergoline for senile dementia or other disorders with vascular origins;

Bromocriptine for pituitary tumors, Parkinson's disease, hyperprolactinaemia, neuroleptic malignant syndrome, or type 2 diabetes; and

Sumatriptan, Zolmitriptan, Rizatriptan, Eletriptan, Naratriptan, Frovatriptan, or Almotriptan for migraines or cluster headaches.

In some embodiments, the composition comprising a serotonergic psychedelic/derivative and ketamine/derivative, a pharmaceutically acceptable salt thereof, or a pharmaceutical composition thereof is included in a therapeutically effective amount.

In some embodiments, the brain disorder is a psychiatric disorder including depression, anxiety, and/or post-traumatic stress disorder. In those embodiments, the method of treating the psychiatric disorder includes administering to a subject in need thereof a therapeutically effective amount of a composition comprising a serotonergic psychedelic/derivative and ketamine/derivative, a pharmaceutically acceptable salt thereof, or a pharmaceutical composition thereof. In some embodiments, the brain disorder is depression. In some embodiments, the brain disorder is anxiety. In other embodiments, the brain disorder is post-traumatic stress disorder.

The psychiatric disorder is a behavioral or mental pattern that may cause suffering or a poor ability to function in life. Such features may be persistent, relapsing and remitting, or occur as a single episode.

Depression is related to a mood disorder involving unusually intense and sustained sadness, melancholia, or despair. Anxiety or fear that interferes with normal functioning may be classified as an anxiety disorder. Commonly recognized categories include specific phobias, generalized anxiety disorder, social anxiety disorder, panic disorder, agoraphobia, obsessive-compulsive disorder and post-traumatic stress disorder.

In some embodiments, the brain disorder is a substance use disorder. In those embodiments, the method of treating the substance use disorder includes administering to a subject in need thereof a therapeutically effective amount of a composition comprising a serotonergic psychedelic/derivative and ketamine/derivative, a pharmaceutically acceptable salt thereof, or a pharmaceutical composition thereof.

Substance use disorder (SUD), also known as drug use disorder, is a condition in which the use of one or more substances leads to a clinically significant impairment or distress. The term “substance” in this context is limited to psychoactive drugs. Addiction and dependence are components of a substance use disorder and addiction represents the most severe form of the disorder.

In other embodiments, the brain disorder is a neurodegenerative disorder including Alzheimer's and/or Parkinson's diseases. In those embodiments, the method of treating the neurodegenerative disorder includes administering to a subject in need thereof a therapeutically effective amount of a composition comprising a serotonergic psychedelic/derivative and ketamine/derivative, a pharmaceutically acceptable salt thereof, or a pharmaceutical composition thereof. In some embodiments, the brain disorder is Alzheimer's disease. In other embodiments, the brain disorder is Parkinson's diseases.

Neurodegeneration is the progressive loss of structure or function of neurons, including death of neurons. Many neurodegenerative diseases including amyotrophic lateral sclerosis, Parkinson's, Alzheimer's, and Huntington's occur as a result of neurodegenerative processes.

The subject can be a living organism suffering from a brain disorder that can be treated by administration of a composition comprising a serotonergic psychedelic/derivative and ketamine/derivative, a pharmaceutically acceptable salt thereof, or a pharmaceutical composition thereof. Non-limiting examples of the living organism include humans, other mammals, for example primates, cows, sheep, goats, horses, dogs, cats, rabbits, rats, mice and the like, and other non-mammalian animals. In some embodiments, the subject is a human. In some embodiments, the subject is an animal including a primate, a cow, a sheep, a goat, a horse, a dog, a cat, a rabbit, a rat, or a mice. In some specific embodiments, the subject is a horse. In some specific embodiments, the subject is a dog. In other specific embodiments, the subject is a cat.

The brain condition or disorder in horses, dogs, or cats can be a psychiatric disorder including depression, anxiety, and/or post-traumatic stress disorder. In these embodiments, the method of treating the psychiatric disorder includes administering to a horse, a dog, or a cat in need thereof a therapeutically effective amount of a composition comprising a serotonergic psychedelic/derivative and ketamine/derivative, a pharmaceutically acceptable salt thereof, or a pharmaceutical composition thereof. In some embodiments, the brain disorder is depression. In some embodiments, the brain disorder is anxiety. In other embodiments, the brain disorder is post-traumatic stress disorder.

In some embodiments, the method includes administering to a subject in need thereof a therapeutically effective amount of the composition comprising a serotonergic psychedelic/derivative and ketamine/derivative, a pharmaceutically acceptable salt thereof, or a pharmaceutical composition thereof.

In some embodiments, the method includes administering a second agent (e.g.

therapeutic agent). In some embodiments, the method includes administering a second agent (e.g. therapeutic agent) in a therapeutically effective amount. In some embodiments, the second agent is an agent for treating a brain condition or disorder. In some embodiments, the second agent is an anti-psychiatric disorder agent. In other embodiments, the second agent is an anti-substance use disorder agent. In other embodiments, the second agent is an anti-neurodegenerative agent.

In some embodiments, the subject is not already being treated with Ergometrine for postpartum hemorrhage and postabortion hemorrhage due to uterine atony.

In some embodiments, the subject is not already being treated with Dihydroergotamine for migraines or cluster headaches.

In some embodiments, the subject is not already being treated with Methyl ergometrine for routine management after delivery of the placenta, postpartum atony and hemorrhage, subinvolution, or migraines.

In some embodiments, the subject is not already being treated with Methysergide for migraines or cluster headaches.

In some embodiments, the subject is not already being treated with Ergotamine for migraines or cluster headaches.

In some embodiments, the subject is not already being treated with Cabergoline for hyperprolactinemic disorders or Parkinson's disease.

In some embodiments, the subject is not already being treated with Pergolide for Parkinson's disease.

In some embodiments, the subject is not already being treated with Lisuride for Parkinson's disease.

In some embodiments, the subject is not already being treated with Nicergoline for senile dementia or other disorders with vascular origins.

In some embodiments, the subject is not already being treated with Bromocriptine for pituitary tumors, Parkinson's disease, hyperprolactinaemia, neuroleptic malignant syndrome, or type 2 diabetes.

In other embodiments, the subject is not already being treated with Sumatriptan for migraines or cluster headaches.

In other embodiments, the subject is not already being treated with Zolmitriptan for migraines or cluster headaches.

In other embodiments, the subject is not already being treated with Rizatriptan for migraines or cluster headaches.

In still other embodiments, the subject is not already being treated with Eletriptan for migraines or cluster headaches.

In still other embodiments, the subject is not already being treated with Naratriptan for migraines or cluster headaches.

In yet other embodiments, the subject is not already being treated with Frovatriptan for migraines or cluster headaches.

In yet other embodiments, the subject is not already being treated with Almotriptan for migraines or cluster headaches.

In one specific embodiment, the method for treating the brain disorder includes administering to a subject in need thereof a therapeutically effective amount of Ergometrine, a pharmaceutically acceptable salt thereof, or a pharmaceutical composition thereof, provided the subject is not already being treated with Ergometrine for postpartum hemorrhage and postabortion hemorrhage due to uterine atony.

### Example Embodiments

Examples of possible embodiments are described below:

1. A composition, comprising:

a psychedelic compound comprising a serotonergic psychedelic compound; and

ketamine.

2. The composition of embodiment 1, wherein the psychedelic compound, drug, or pharmaceutical is chosen from a tryptamine, phenethylamine, or lysergamide.

3. The composition of embodiment 1, wherein the psychedelic compound, drug, or pharmaceutical is chosen from psilocybin, psilocin, or a psilocybin derivative.

4. A composition according to embodiments 1-3, wherein the ketamine is S-ketamine.

5. A composition according to embodiments 1-4, wherein the ketamine is S-ketamine hydrochloride.

6. A composition according to embodiments 1-5, wherein the concentration of the ketamine is at least 110 mg/mL of total composition volume.

7. A composition according to embodiments 1-5, wherein the concentration of the ketamine is at least 125 mg/mL, based on the total volume of the composition.

8. A composition according to embodiments 1-5, wherein the concentration of the ketamine is at least 130 mg/mL, based on the total volume of the composition.

9. A composition according to embodiments 1-5, wherein ketamine is present in a concentration in the range of eq. 125 mg/mL to eq. 250 mg/mL, based on the total volume of the composition.

10. A composition according to embodiments 1-5, wherein ketamine is present in a concentration in the range of eq. 125 mg/mL to eq. 180 mg/mL, based on the total volume of the composition.

11. A composition according to embodiments 1-5, wherein ketamine is present in a concentration in the range of eq. 125 mg/mL to eq. 150 mg/mL, based on the total volume of the composition.

12. A composition according to embodiments 1-5, wherein ketamine is present in a concentration in the range of eq. 126 mg/mL to eq. 162 mg/mL, based on the total volume of the composition.

13. The composition of embodiments 1-5, wherein the ketamine is present at a concentration in the range of 125 mg/mL equivalents to 200 mg/mL equivalents based on the total volume of the composition; or the ketamine is present at a concentration in the range of 125 mg/mL equivalents to 180 mg/mL equivalents based on the total volume of the composition; or wherein the ketamine is present at a concentration in the range of 125 mg/mL equivalents to 150 mg/mL equivalents based on the total volume of the composition.

14. A composition according to embodiments 1-12, further comprising a buffering agent.

15. A composition according to embodiments 1-14, wherein the composition has a pH value within a range from 3.5 to 6.5.

16. A composition according to embodiments 1-15, wherein the composition has a pH value within a range from 4.0 to 6.5.

17. A composition according to embodiments 1-16, wherein the composition has a pH value within a range from 4.0 to 5.5.

18. A composition according to embodiments 1-15, wherein the composition has a pH value within a range from 3.5 to 5.5.

19. A composition according to embodiments 1-18, further comprising morphine.

20. A composition according to embodiments 1-19, further comprising a cannabinoid.

21. A composition according to embodiments 1-20, wherein the buffering agent is sodium hydroxide.

22. A composition according to embodiments 1-21, which is suitable for nasal administration.

23. A composition according to embodiments 1-21, which is suitable for intravenous administration.

24. A composition according to embodiments 1-22, for use in the treatment of depression.

25. A composition according to embodiments 1-22, for use in the treatment of post traumatic stress disorder.

26. A composition according to embodiments 1-22, for use in the treatment of anxiety.

27. A composition according to embodiments 1-22, for use in the treatment of mental health disorder.

28. A composition according to embodiments 1-27, wherein the psychedelic compound, drug, or pharmaceutical and the ketamine are present in the composition in a molar ratio of between 100:1 to 1:100.

29. A composition according to embodiments 1-27, wherein the molar ratio is between 75:1 to 1:75.

30. A composition according to embodiments 1-27, wherein the molar ratio is between 50:1 to 1:50.

31. A composition according to embodiments 1-27, wherein the molar ratio is between 25:1 to 1:25.

32. A composition according to embodiments 1-27, wherein the molar ratio is between 10:1 to 1:10.

33. A composition according to embodiments 1-27, wherein the molar ratio is between 5:1 to 1:5.

34. A composition according to embodiments 1-27, wherein the psychedelic compound, drug, or pharmaceutical and the ketamine are present as a homogeneous mixture within the composition.

35. A composition, comprising:

psilocybin, psilocin, or a psilocybin derivative; and

ketamine.

36. A composition, comprising: psilocybin, psilocin, or a psilocybin derivative;

morphine; and ketamine.

37. A composition, comprising: a first part comprising a first composition comprising psilocybin, psilocin, or a psilocybin derivative; and a second part comprising a second composition comprising ketamine or a ketamine derivative.

38. A pharmaceutical composition, comprising:

psilocybin or a psilocybin derivative;

S-ketamine hydrochloride; and

water.

39. A pharmaceutical composition, comprising:

psilocybin or a psilocybin derivative;

S-ketamine hydrochloride;

morphine; and

water.

40. A pharmaceutical composition, comprising:

psilocybin or a psilocybin derivative;

S-ketamine hydrochloride; and

water.

41. A method for increasing neural plasticity, comprising contacting a neuronal cell with a composition, in an amount sufficient to increase neural plasticity of the neuronal cell, wherein the composition produces a maximum number of dendritic crossings with an increase of greater than 1.0 fold by a Sholl Analysis.

42. The method of embodiment 41, wherein the composition produces an area-under-curve (AUC) of a Sholl plot with an increase of greater than 1.0 fold.

43. The method of embodiments 41, wherein the composition produces a number of dendritic branches with an increase of greater than 1.0 fold.

44. The method of embodiment 41, wherein the composition produces a total dendritic length with an increase of greater than 1.0 fold.

45. The method of embodiment 41, wherein the composition produces a density of dendritic spines with an increase of greater than 1.0 fold.

46. The method of embodiment 41, wherein the composition produces a density of synapses with an increase of greater than 1.0 fold.

47. The method of embodiment 46, wherein the composition produces a density of a presynaptic protein with an increase of greater than 1.0 fold, wherein the presynaptic protein is Vesicular glutamate transporter 1 (VGLUT1).

48. The method of embodiment 41, wherein the composition increases at least one of translation, transcription, and secretion of neurotrophic factors.

49. The method of embodiment 48, wherein the neurotrophic factor is at least one of a brain-derived neurotrophic factor (BDNF) and a glial cell line-derived neurotrophic factor (GDNF).

50. The method of embodiment 49, wherein the translation of the brain-derived neurotrophic factor (BDNF) has an increase of greater than 1.0 fold.

51. The method of embodiment 41, wherein the composition is a blood-brain-barrier (BBB) penetrator.

52. The method of embodiment 41, wherein the composition has a pKa of from 7.0 to 10.0.

53. The method of embodiment 52, wherein the composition is permeable across cell membranes.

54. The method of embodiments 41-53, wherein the composition comprises a first compound that is a serotonergic psychedelic, the first compound is chosen from a tryptamine, phenethylamine, or lysergamide.

55. The method of embodiments 41-53, wherein the composition comprises a first compound that is a serotonergic psychedelic, the first compound is chosen from psilocybin, psilocin, or a psilocybin derivative.

56. The method of embodiments 41-55, wherein the composition comprises a second compound selected from the group consisting of ketamine, and a ketamine derivative.

57. The method of embodiments 41-55, wherein the composition comprises a second compound selected from the group consisting of S-ketamine.

58. A method of treating a brain disorder, comprising administering to a subject in need thereof a therapeutically effective amount of a composition, thereby treating the brain disorder, wherein the composition increases neural plasticity of the neuronal cell;

provided that the subject is not already being treated with one or more of following:

Ergometrine for postpartum hemorrhage and postabortion hemorrhage due to uterine atony; the subject is not treated with Dihydroergotamine for migraines or cluster headaches;

Methylergometrine for routine management after delivery of the placenta, postpartum atony and hemorrhage, subinvolution, or migraines;

Methysergide for migraines or cluster headaches; Ergotamine for migraines or cluster headaches;

Cabergoline for hyperprolactinemic disorders or Parkinson's disease; Pergolide for Parkinson's disease;

Lisuride for Parkinson's disease;

Nicergoline for senile dementia or other disorders with vascular origins;

Bromocriptine for pituitary tumors, Parkinson's disease, hyperprolactinaemia, neuroleptic malignant syndrome, or type 2 diabetes; or

Sumatriptan, Zolmitriptan, Rizatriptan, Eletriptan, Naratriptan, Frovatriptan, or Almotriptan for migraines or cluster headaches.

59. The method of embodiment 58, wherein the brain disorder is a psychiatric disorder selected from the group consisting of depression, anxiety, and post-traumatic stress disorder.

60. The method of embodiment 58, wherein the brain disorder is a substance use disorder.

61. The method of embodiment 58, wherein the brain disorder is a neurodegenerative disorder selected from the group consisting of Alzheimer's and Parkinson's diseases.

62. A pharmaceutical composition, comprising:

psilocybin or a psilocybin derivative;

S-ketamine hydrochloride;

a cannabinoid; and

water.

Where a conflict exists between the instant application and a reference provided herein, the instant application shall dominate.

All of the above U.S. patents, U.S. patent application publications, U.S. patent applications, foreign patents, foreign patent applications and non patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention.

All of the compositions and/or methods disclosed and claimed herein can be made and executed without undue experimentation in light of the present disclosure. While the compositions and methods of this invention have been described in terms of specific embodiments, it will be apparent to those of skill in the art that variations of the compositions and/or methods and in the steps or in the sequence of steps of the method described herein can be made without departing from the concept, spirit and scope of the invention. More specifically, it will be apparent that certain agents which are both chemically and physiologically related may be substituted for the agents described herein while the same or similar results are achieved. All such similar substitutes and modifications apparent to those skilled in the art are deemed to be within the spirit, scope and concept of the invention as defined by the appended claims.

The references cited herein throughout, to the extent that they provide exemplary procedural or other details supplementary to those set forth herein, are all specifically incorporated herein by reference. Furthermore, referenced cited herein, references cited within those references cited herein, and so forth, are all specifically incorporated herein by reference. At certain points throughout the specification, references are referred to using a number in square brackets. Those numbers correspond to the following list of references, each of which is incorporated herein by reference:

### REFERENCES

1. Charney et al. (2020). Method of treating post-traumatic stress disorder (US Patent Application No. US 2020/0147006A1). U.S. Patent and Trademark Office.
2. Kai Zhang & Kenji Hashimoto (2019) An update on ketamine and its two enantiomers as rapid-acting antidepressants, Expert Review of Neurotherapeutics, 19:1, 83-92, DOI: 10.1080/14737175.2019.1554434
3. Sanacora G, Frye M A, McDonald W, Mathew S J, Turner M S, Schatzberg A F, et al. (April 2017). “A Consensus Statement on the Use of Ketamine in the Treatment of Mood Disorders”. JAMA Psychiatry. 74 (4): 399-405. doi:10.1001/jamapsychiatry.2017.0080.
4. Molero P, Ramos-Quiroga J A, Martin-Santos R, Calvo-Sanchez E, Gutierrez-Rojas L, Meana J J (May 2018). “Antidepressant Efficacy and Tolerability of Ketamine and Esketamine: A Critical Review”. CNS Drugs. 32 (5): 411-420. doi:10.1007/s40263-018-C519-3
5. Singh I, Morgan C, Curran V, Nutt D, Schlag A, McShane R (May 2017). “Ketamine treatment for depression: opportunities for clinical innovation and ethical foresight”. The Lancet. Psychiatry. 4 (5): 419-426. doi:10.1016/S2215-0366(17)30102-5
6. Marcantoni W S, Akoumba B S, Wassef M, Mayrand J, Lai H, Richard-Devantoy S, Beauchamp S (December 2020). “A systematic review and meta-analysis of the efficacy of intravenous ketamine infusion for treatment resistant depression: January 2009-January 2019”. J Affect Disord. 277: 831-841. doi:10.1016/j.jad.2020.09.007
7. Swainson J, McGirr A, Blier P, Brietzke E, Richard-Devantoy S, Ravindran N, Blier J, Beaulieu S, Frey B N, Kennedy S H, McIntyre R S, Milev R V, Parikh S V, Schaffer A, Taylor V H, Tourjman V, van Ameringen M, Yatham L N, Ravindran A V, Lam R W (November 2020). “The Canadian Network for Mood and Anxiety Treatments (CANMAT) Task Force Recommendations for the Use of Racemic Ketamine in Adults with Major Depressive Disorder: Recommandations Du Groupe De Travail Du Réseau Canadien Pour Les Traitements De L'humeur Et De L'anxiété (Canmat) Concernant L'utilisation De La Kétamine Racémique Chez Les Adultes Souffrant De Trouble Dépressif Majeur”. Can J Psychiatry: 706743720970860. doi:10.1177/0706743720970860.
8. Bobo W V, Riva-Posse P, Goes F S, Parikh S V (April 2020). “Next-Step Treatment Considerations for Patients With Treatment-Resistant Depression That Responds to Low-Dose Intravenous Ketamine”. Focus (Am Psychiatr Publ). 18 (2): 181-192. doi:10.1176/appi.focus.20190048
9. Gass, N., Becker, R., Reinwald, J. et al. Differences between ketamine's short-term and long-term effects on brain circuitry in depression. Transl Psychiatry 9, 172 (2019). https://doi.org/10.1038/s41398-019-C506-6
10. Bobo W V, Riva-Posse P, Goes F S, Parikh S V (April 2020). “Next-Step Treatment Considerations for Patients With Treatment-Resistant Depression That Responds to Low-Dose Intravenous Ketamine”. Focus (Am Psychiatr Publ). 18 (2): 181-192. doi:10.1176/appi.focus.20190048
11. Charney et al. (2014). Intranasal Administration of Ketamine to Treat Depression (U.S. Pat. No. 8,785,500 B2). U.S. Patent and Trademark Office.
12. Soumier A, Carter R M, Schoenfeld T J, Cameron H A. “New Hippocampal Neurons Mature Rapidly in Response to Ketamine But Are Not Required for Its Acute Antidepressant Effects on Neophagia in Rats.” eNeuro. 2016; 3(2):ENEURO.0116-15.2016. Published 2016 Mar. 31. doi:10.1523/ENEURO.0116-15.2016
13. Jamie Sleigh, Martyn Harvey, Logan Voss, Bill Denny. “Ketamine—More mechanisms of action than just NMDA blockade, Trends in Anaesthesia and Critical Care, Volume 4, Issues 2-3, 2014, Pages 76-81,

ISSN 2210-8440, https://doi.org/10.1016/j.tacc.2014.03.002.
14. Ronald S. Duman, Nanxin Li, Rong-Jian Liu, Vanja Duric, George Aghajanian, “Signaling pathways underlying the rapid antidepressant actions of ketamine, Neuropharmacology,” Volume 62, Issue 1,

2012, Pages 35-41, ISSN 0028-3908, https://doi.org/10.1016/j.neuropharm.2011.08.044.
15. Ly C, Greb A C, Cameron L P, et al. “Psychedelics Promote Structural and Functional Neural Plasticity.” Cell Rep. 2018; 23(11):3170-3182. doi:10.1016/j.celrep.2018.05.022
16. Chadeayne (2021). Compositions and methods comprising a psilocybin derivative (U.S. Pat. No. 10,933,073 B2). U.S. Patent and Trademark Office.
17. Möller, I. R., Slivacka, M., Nielsen, A. K. et al. Conformational dynamics of the human serotonin transporter during substrate and drug binding. Nat Commun 10, 1687 (2019). https://doi.org/10.1038/s41467-019-09675-z
18. Coleman, J., Green, E. & Gouaux, E. X-ray structures and mechanism of the human serotonin transporter. Nature 532, 334-339 (2016). https://doi.org/10.1038/nature17629
19. Jumper, J et al. Highly accurate protein structure prediction with AlphaFold. Nature (2021).
20. ACS Chem. Neurosci. 2013, 4, 2, 295-309. Publication Date: Oct. 24, 2012. https://doi.org/10.1021/cn300148r
21. Morris, G. M., Huey, R., Lindstrom, W., Sanner, M. F., Belew, R. K., Goodsell, D. S. and Olson, A. J. (2009) Autodock4 and AutoDockTools4: automated docking with selective receptor flexibility. J. Computational Chemistry 2009, 16: 2785-91.
22. O. Trott, A. J. Olson, AutoDock Vina: improving the speed and accuracy of docking with a new scoring function, efficient optimization and multithreading, Journal of Computational Chemistry 31 (2010) 455-461
23. David A. Drachman, “Do we have brain to spare?” Neurology June 2005, 64 (12) 2004-2005; DOI: 10.1212/01.WNL.0000166914.38327.BB
24. Crisp K M, Sutter E N, Westerberg J A. Pencil-and-Paper Neural Networks: An Undergraduate Laboratory Exercise in Computational Neuroscience. J Undergrad Neurosci Educ. 2015; 14(1):A13-A22. Published 2015 Oct. 15.
25. McCulloch W S, Pitts W H (1943) A logical calculus of the ideas immanent in nervous activities. Bull Math Biophys 5:115-133.
26. Hopfield J J (1982) Neural networks and physical systems with emergent collective computational abilities. PNAS 79:2554-2558.

Those skilled in the art will recognize, or be able to ascertain using no more than routine experimentation, many equivalents to the specific embodiments described herein. The scope of the present invention is not intended to be limited to the Description or the details set forth therein. Articles such as “a”, “an” and “the” may mean one or more than one unless indicated to the contrary or otherwise evident from the context. Claims or descriptions that include “or” or “and/or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the claims (whether original or subsequently added claims) is introduced into another claim (whether original or subsequently added). For example, any claim that is dependent on another claim can be modified to include one or more element(s), feature(s), or limitation(s) found in any other claim, e.g., any other claim that is dependent on the same base claim. Any one or more claims can be modified to explicitly exclude any one or more embodiment(s), element(s), feature(s), etc. For example, any particular sideroflexin, sideroflexin modulator, cell type, cancer type, etc., can be excluded from any one or more claims.

Where elements are presented as lists, e.g., in Markush group format, each subgroup of the elements is also disclosed, and any element(s) can be removed from the group. For purposes of conciseness only some of these embodiments have been specifically recited herein, but the present disclosure encompasses all such embodiments. It should also be understood that, in general, where the invention, or aspects of the invention, is/are referred to as comprising particular elements, features, etc., certain embodiments of the invention or aspects of the invention consist, or consist essentially of, such elements, features, etc.

Where numerical ranges are mentioned herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. Where phrases such as “less than X”, “greater than X”, or “at least X” is used (where X is a number or percentage), it should be understood that any reasonable value can be selected as the lower or upper limit of the range. It is also understood that where a list of numerical values is stated herein (whether or not prefaced by “at least”), the invention includes embodiments that relate to any intervening value or range defined by any two values in the list, and that the lowest value may be taken as a minimum and the greatest value may be taken as a maximum. Furthermore, where a list of numbers, e.g., percentages, is prefaced by “at least”, the term applies to each number in the list. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments 5% or in some embodiments 10% of a number in either direction (greater than or less than the number) unless otherwise stated or otherwise evident from the context (e.g., where such number would impermissibly exceed 100% of a possible value).

Although the present invention has been described with reference to preferred embodiments, it will be understood by those skilled in the art that various changes in form and detail may be made without departing from the scope of the present invention as set forth in the accompanying claims.

What is claimed is:

1. A composition comprising a serotonergic psychedelic compound and a ketamine compound in synergistically effective amounts for treating a patient suffering from a brain condition or disorder and/or promoting neural plasticity in a patient in need thereof.

2. The composition of claim 1, wherein the serotonergic psychedelic compound is selected from the group consisting of psilocybin, psilocin, a psilocybin derivative, tryptamine, phenethylamine, lysergamide, and one or more combinations thereof.

3. The composition of claim 1, wherein the psychedelic compound is selected from the group consisting of psilocybin, psilocin, and a psilocybin derivative.

4. The composition according to claim 1, wherein the ketamine compound is S-ketamine.

5. The composition according to claim 1, wherein the ketamine compound is S-ketamine hydrochloride.

6. A composition according to claim 1, wherein the brain condition or disorder is depression.

7. A method of treating a patient suffering from a brain condition or disorder and/or promoting neural plasticity in a patient in need thereof comprising administering to the patient a composition according to claim 1.

8. The method of claim 7, wherein ketamine is administered at a dosage of between about 0.13 and about 0.53 mg/kg/day.

9. The method of claim 7, wherein the administration of multiple doses of said composition over a period of 7 days.

10. The method of claim 7, which comprises administration of a single dose of said composition over a period of 7 days.

11. The method of claim 7, wherein said psychedelic compound is selected from the group consisting of from the group consisting of psilocybin, psilocin, a psilocybin derivative, tryptamine, phenethylamine, lysergamide, and one or more combinations thereof.

12. The method of claim 7, wherein the brain condition or disorder comprises a major depressive disorder.

13. The method of claim 7, wherein up to 250 mg/day of ketamine is administered to the patient.

14. The method of claim 7, wherein the patient is administered the composition after not responding to at least two antidepressant trials.

15. The method of claim 7, further comprising administering multiple doses of the composition to the patient.

16. The method of claim 7, wherein the dosage amount of ketamine ranges between about 0.1 mg/kg/day to about 3.0 mg/kg/day.

17. The method of claim 7, wherein the symptoms of said depression are alleviated within 2 hours of administering the ketamine.

18. The method of claim 7, wherein the symptoms of the depression are alleviated within one day of administering the composition.

19. A method of making a composition of claim 1 comprising presenting a synergistically effective amount of a serotonergic psychedelic compound; presenting a synergistically effective amount of a ketamine compound; and combining the serotonergic psychedelic compound and the ketamine compound in a pharmaceutically acceptable dosage form for treating a patient suffering from a brain condition or disorder and/or promoting neural plasticity in a patient in need thereof.

20. The composition of claim 1, further comprising morphine.
